(19) United States
(12) Patent Application Publication
(10) Pub. No.: US 2023/0167439 A1
(43) Pub. Date: Jun. 1, 2023

(54) METHOD FOR PREPARING NUCLEIC ACID STRUCTURE

(71) Applicant: SEOUL NATIONAL UNIVERSITY R&DB FOUNDATION, Seoul (KR)

(72) Inventors: Do Nyun KIM, Seoul (KR); Myoung Seok KIM, Seoul (KR)

(21) Appl. No.: 18/070,646

(22) Filed: Nov. 29, 2022

(30) Foreign Application Priority Data
Nov. 29, 2021 (KR) 10-2021-0167625

(51) Int. Cl. C12N 15/11

(52) U.S. Cl. CPC C12N 15/11

(57) ABSTRACT

A method for preparing a nucleic acid structure capable of being repeatedly folded and unfolded in various directions uses a nucleic acid wireframe having a plurality of line segments. The method implements various types of nucleic acid origami on the nanoscale by designing a crease pattern along the line segment of the nucleic acid wireframe.

### CROSS REFERENCE TO RELATED APPLICATIONS AND CLAIM OF PRIORITY

This application claims the benefit under 35 USC §119 of Korean Patent Application No. 10-2021-0167625, filed on Nov. 29, 2021, in the Korea Intellectual Property Office, the entire disclosure of which is incorporated herein by reference in its entirety.

### BACKGROUND

### 1. Field of the Invention

The present invention relates to a nucleic acid structure capable of being repeatedly folded and unfolded in various directions and a preparation method thereof, which may be utilized in nanoprocess and pharmaceutical fields by implementing an origami method using nucleic acid, which is a biomaterial, on the nanoscale.

In addition, the present invention relates to a method for detecting a target nucleic acid using a nucleic acid structure capable of being repeatedly folded and unfolded in various directions, which may be utilized in a biomolecular diagnostic system field.

### 2. Description of the Related Art

Structural DNA origami technique is a technique to design a nanostructure with atomic-level precision by using self-assembled DNA strands, and specifically, refers to a technique for synthesizing a DNA nanostructure having a desired shape by ergonomically designing complementary base pairing of a single very long DNA strand, a scaffold and numerous relatively short DNA strands, and staples, which form a template of the nanostructure.

Origami technique is an engineering technique using origami, and has been generally used to produce a three-dimensional structure having various shapes or characteristics by designing a variety of crease patterns in a flat structure.

However, the above-described method has problems in that it is necessary to design and synthesize a new structure each time in order to use various types of structures, or it is difficult to provide a desired modification to the structure due to internal rigidity of the structure.

Thereby, the present inventors have made best efforts to develop a core mechanism that can transform one structure into various shapes, and as a result, be able to construct a DNA structure in a flexible wireframe shape and design a crease pattern that can be folded in a specific direction to implement a structure capable of being repeatedly and completely folded and unfolded in various shapes and a preparation method thereof, and have completed the present invention.

### SUMMARY

It is an object of the present invention to provide a method for preparing a nucleic acid structure capable of being repeatedly folded and unfolded in various directions.

In addition, another object of the present invention is to provide a nucleic acid structure capable of being repeatedly folded and unfolded in various directions.

Further, another object of the present invention is to provide a method for detecting a target nucleic acid using the nucleic acid structure capable of being repeatedly folded and unfolded in various directions.

To achieve the above objects, the following technical solutions are adopted in the present invention.

1. A method for preparing a nucleic acid structure, including: a first step of binding at least a pair of first single-stranded nucleic acids protruding in the same direction to portions symmetric with respect to at least one crease line segment, respectively, in a nucleic acid wireframe having a plurality of line segments; and a second step of treating the nucleic acid wireframe with a second single-stranded nucleic acid which includes a first portion including a complementary sequence to the pair of first single-stranded nucleic acids and a second portion including a non-complementary sequence to the pair of first single-stranded nucleic acids at a terminal of the first portion, thus to bind the pair of first single-stranded nucleic acids to the second single-stranded nucleic acid.

2. The method for preparing a nucleic acid structure according to the above 1, wherein the pair of first single-stranded nucleic acids consist of two single-stranded nucleic acids having different sequences.

3. The method for preparing a nucleic acid structure according to the above 1, wherein in the first step, the pair of first single-stranded nucleic acids are bound orthogonal to the portions symmetric with respect to the crease line segments in the same direction.

4. The method for preparing a nucleic acid structure according to the above 1, including a plurality of crease line segments, wherein the pairs of first single-stranded nucleic acids have different sequences for each of the crease line segments.

5. The method for preparing a nucleic acid structure according to the above 1, wherein in the first step, a plurality of the pair of first single-stranded nucleic acids are bound to the symmetrical portions, respectively, and in the second step, the treating is performed so that a plurality of second single-stranded nucleic acids are bound to the first single-stranded nucleic acids as many as the number thereof.

6. The method for preparing a nucleic acid structure according to the above 1, wherein in the second step, a pair of first single-stranded nucleic acids on both sides of the symmetrical portion are bound to the first portion of the second single-stranded nucleic acid, respectively.

7. The method for preparing a nucleic acid structure according to the above 1, further including the step of binding a fluorescent material and a quencher respectively to both sides of the wireframe so as to be symmetric with respect to the crease line segment, before the second step.

8. The method for preparing a nucleic acid structure according to the above 1, further including a third step of adding a third single-stranded nucleic acid which includes a complementary sequence to the first portion and the second portion, thus to bind the second single-stranded nucleic acid to the third single-stranded nucleic acid so as to be separated from the first single-stranded nucleic acid.

9. A method for detecting a target nucleic acid, including: a first step of binding at least a pair of first single-stranded nucleic acids protruding in the same direction to portions symmetric with respect to at least one crease line segment, respectively, in a nucleic acid wireframe having a plurality of line segments; and a second step of treating the nucleic acid wireframe with a sample suspected of containing a target nucleic acid which includes a first portion including a complementary sequence to the pair of first single-stranded nucleic acids and a second portion including a non-complementary sequence to the pair of first single-stranded nucleic acids at a terminal of the first portion, thus to confirm whether the wireframe is folded at the line segment.

10. The method for detecting a target nucleic acid according to the above 9, wherein the pair of first single-stranded nucleic acids consist of two single-stranded nucleic acids having different sequences.

11. The method for detecting a target nucleic acid according to the above 9, including a plurality of crease line segments, wherein the pairs of first single-stranded nucleic acids have different sequences for each of the crease line segments.

12. The method for detecting a target nucleic acid according to the above 11, wherein the target nucleic acid includes a plurality of nucleic acids having different sequences.

13. The method for detecting a target nucleic acid according to the above 9, further including the step of binding a fluorescent material and a quencher respectively to both sides of the wireframe so as to be symmetric with respect to the crease line segment, before the second step.

14. The method for detecting a target nucleic acid according to the above 9, wherein, when the line segment is folded, it is determined that the target nucleic acid corresponding to the pair of first single-stranded nucleic acids which are bound corresponding to the folded line segment is present in the sample.

15. The method for detecting a target nucleic acid according to the above 14, wherein when a fluorescence intensity of the fluorescent material is decreased, it is determined that the line segment is folded.

16. The method for detecting a target nucleic acid according to the above 13, including a plurality of crease line segments, and an amount of the target nucleic acid in the sample is quantified based on a degree of a decrease in the fluorescence intensity of the fluorescent material.

17. A nucleic acid structure including: a nucleic acid wireframe having a plurality of line segments; and at least a pair of first single-stranded nucleic acids which protrude in the same direction and are bound to portions symmetric with respect to at least one crease line segment in the nucleic acid wireframe, wherein among a first portion and a second portion adjacent to each other in a target nucleic acid sequence, the pair of first single-stranded nucleic acids have a complementary sequence to at least a part of the first portion and a non-complementary sequence to the second portion.

18. The nucleic acid structure according to the above 17, wherein the pair of first single-stranded nucleic acids consist of two single-stranded nucleic acids having different sequences.

19. The nucleic acid structure according to the above 17, wherein the nucleic acid wireframe includes a DNA origami wireframe.

20. The nucleic acid structure according to the above 17, wherein a fluorescent material and a quencher are further bound respectively to both sides of the wireframe so as to be symmetric with respect to the crease line segment.

When using the preparation method of the present invention, it is possible to prepare a nucleic acid structure capable of being repeatedly folded and unfolded in various directions.

The preparation method and structure of the present invention use a flexible nucleic acid wireframe structure, such that it is possible to completely fold and unfold the structure.

When using the detection method of the present invention, it is possible to detect a target nucleic acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and other advantages of the present invention will be more clearly understood from the following detailed description taken in conjunction with the accompanying drawings, in which:

FIG. 1A to FIG. 1F schematically illustrate a design of DNA origami, wherein a scale-bar indicates 100 nm;

FIG. 2A to FIG. 2B illustrate synthesis of a DNA wireframe paper. Specifically, (a) shows results of gel electrophoresis using 1.5% by weight (“wt.%”) of agarose gel stained with ethidium-bromide for 90 minutes at 75 V by changing a cation concentration. As a result, square (SQ) and hexagonal (HX)-shaped DNA papers were synthesized using the cation concentration of 12 mM MgCl₂. (b) is representative atomic force microscopy (AFM) images of SQ and HX under 12 mM MgCl₂ annealing conditions, wherein scale-bars indicate 500 nm and 100 nm;

FIG. 3A to FIG. 3B illustrate a procedure for estimating an origami yield based on the AFM images. Specifically, (a) illustrates image processing for estimation of origami yield using an example of SQ Q1 origami. Herein, all particles in a raw AFM image (left) were systematically numbered (middle), filtered according to a size to remove aggregated particles, and renumbered using customizable MATLAB code (right), wherein a scale-bar indicates 1 µm. In (b), particles which are partially displayed or have illogical shapes (diagonal strikethroughs) were excluded from the estimation of origami yield, and individual images with numbers indicate intended shapes of monomers, and dotted numbers indicate unintended shapes of monomers, wherein a scale-bar indicates 100 nm;

FIG. 4A to FIG. 4B are schematic diagram for describing an origami-dependent fluorescence intensity control design. Specifically, (a) illustrates a crease pattern having 3 pairs of SQ H1 with two quenchers Q and reporters R, (b) illustrates a crease pattern having 3 pairs of SQ H2 with two quenchers Q and reporters R, (c) illustrates a schematic diagram of a reporter quenched in SQ H1, and (d) illustrates a schematic diagram of a reporter quenched in SQ H2;

FIG. 5A to FIG. 5B illustrate crease handle designs for folding in mountain and valley shapes. Specifically, (a) illustrates a conceptual design of folding in the mountain shape using the example of SQ Q1, and (b) illustrates a conceptual design of folding in the valley shape using the example of SQ Q1;

FIG. 6A to FIG. 6B illustrate representative AFM images of nanoscale origami. Specifically, (a) a scale-bar indicates 1 µm, and (b) is exemplary AFM images of SQ H1 origami, wherein a scale-bar indicates 100 nm;

FIG. 7A to FIG. 7E are diagrams illustrating origami implemented in various nanoscales. Specifically, (a) is representative AFM images illustrating two types of wireframe DNA paper, which include square (SQ) and hexagonal (HX)-shaped crease patterns. Herein, H denotes half fold, Q denotes quarter fold, and suffix numbers denotes the fold numbers, wherein a scale-bar indicates 100 nm. (b) is schematic diagrams of nanoscale origami and AFM images illustrating results thereof, wherein six types of SQ origami and three types of HX origami were implemented. Herein, wide arrows indicate a process of adding glue strands, wherein a scale-bar indicates 50 nm. (c) illustrates finite element simulation results of nanoscale origami conducted in SNUPI, and (d) illustrates results of gel electrophoresis using 1.5 wt.% of agarose gel stained with ethidium-bromide (EtBr) of folded DNA paper. As a result, a variety of migrations according to the programmed crease pattern were observed. Herein, each dotted arrow indicates band positions on the DNA paper unfolded as a reference, wherein L denotes a ladder;

FIG. 8 illustrates crease patterns of SQ and HX in detail. Herein, segments with arrows indicate DNA segments with 3′ and 5′ crease handles which bind to glue 1 strand (G1), respectively, and dark horizontal segments in SQ H1 (G2) &H2 (G1) indicate DNA segments with 3′ and 5′ crease handles which bind to glue 2 strand(G2), respectively.

FIG. 9A to FIG. 9C are exemplary AFM images of SQ origami (I), (II) and HX origami wherein a scale-bar indicates 100 nm;

FIG. 10A to FIG. 10C illustrate SNUPI simulation results for equilibrium configurations of the SQ origami (I), (II), and HX origami wherein it was assumed that the glue strand and the designed crease handle were completely bound to each other;

FIG. 11 illustrates results of gel electrophoresis of folded DNA papers. Specifically, (a) and (b) show the results of gel electrophoresis using 1.5 wt.% of agarose gel stained with ethidium-bromide for 90 minutes at 75 V in order to compare the migration of two types of folded DNA papers, SQ and HX origami. Herein, each relative frontal value was measured at a relative distance between an entrance and a band position;

FIG. 12A to FIG. 12D illustrate the optimization of origami yield. Specifically, (a) indicates the number of pairs of crease handles, wherein a scale-bar indicates 100 nm, and (b) illustrates the mechanical relaxation of the folding portion. Originally, SQ consisted of 4 stiff regions (light boxes) in the nicked state and 4 flexible regions (dark boxes) in the unpaired state. The left lower graph shows normalized flexural rigidities of DNA papers with gaps from nick (0-gap) to 5-gap. (c) is a graph illustrating yield results of SQ H1 origami by changing both the number of pairs and gaps, and (d) is a graph illustrating optimal yield results for representative five types of single-folded origami. In (a, c and d), at least three AFM measurements were performed to estimate yield and standard deviation;

FIG. 13A is representative AFM images (1 pair, 90 degrees, gap) of SQ H1;

FIG. 13B is representative AFM images (1 pair, 180 degrees, gap) of SQ H1;

FIG. 13C is representative AFM images (two pairs, gap) of SQ H1;

FIG. 13D is representative AFM images (3 pairs, gap) of SQ H1;

In FIG. 13A to FIG. 13D, each origami was designed by varying a length of the gap at four folded points (boxes on the DNA paper), and at least two AFM measurements were performed to estimate the origami yield. Herein, dark lines indicate DNA segments modified with 3′ and 5′ crease handles, respectively, and wide arrows indicate the process of adding glue strands, wherein a scale-bar indicates 1 µm;

FIG. 13E is representative AFM images (pairs, 4-nt unpaired) for SQ H2, wherein each origami was designed by varying the number of pairs of segments along an H2 crease, and at least three AFM measurements were performed to estimate the origami yield and standard deviation. Herein, a scale-bar indicates 1 µm;

FIG. 13F is representative AFM images (pairs, gap) of SQ Q1, wherein each origami was designed by varying the number of pairs and gaps (boxes) along a folding line, and at least three AFM measurements were performed to estimate the origami yield and standard deviation. Herein, a scale-bar indicates 1 µm;

FIG. 13G is a representative AFM image of SQ Q12 (1 pair, nick);

FIG. 13H is a representative AFM image (1 pair, nick) of SQ Q13;

FIG. 13I is a representative AFM image (1 pair, nick) of SQ Q123;

FIG. 13J is a representative AFM image (1 pair, nick) of SQ Q1234;

In FIG. 13G to FIG. 13J, at least three AFM measurements were performed to estimate the origami yield. Herein, a scale-bar indicates 1 µm;

FIG. 14A is representative AFM images (pairs, 5-nt unpaired) for HX H1, wherein each origami was designed by varying the number of pairs of segments along an H1 crease, and at least three AFM measurements were performed to estimate the origami yield and standard deviation. Herein, a scale-bar indicates 1 µm;

FIG. 14B is representative AFM images (3 pairs, gap) of HX Q1, wherein each origami was designed by varying the length of the gap at two folding points (circles), and at least three AFM measurements were performed to estimate the origami yield and standard deviation. Herein, a scale-bar indicates 1 µm;

FIG. 14C is representative AFM images (3 pairs, nick) of HX Q12, wherein five AFM measurements were performed to estimate the origami yield. Herein, a scale-bar indicates 1 µm;

FIG. 14D illustrates multi-quarter folding ratios (3 pairs, nick) of HX;

FIG. 15A to FIG. 15C illustrate mechanical crease points of wireframe DNA papers. Specifically, (a) illustrates a crease point configuration of square DNA paper (left), 4 stiff regions with a nick (middle), and 4 flexible regions with 4-nt long unpaired ssDNA (right), (b) illustrates a crease point configuration of hexagonal DNA paper (left), 6 stiff regions with a nick (middle), and 6 flexible regions with 5-nt long unpaired ssDNA (right), and (c) illustrates a modular design of staple strands for replacing nicks with gaps having various lengths;

FIG. 16A to FIG. 16D illustrate verification of nanoscale origami properties. Specifically, (a) shows an orthogonal origami, and the upper view is an orthogonal origami configuration diagram of SQ with H1 and H2 crease patterns. The lower view is representative AFM images for each dominant morphology, wherein a scale-bar indicates 50 nm. (b) shows repeatable origami, wherein the upper view is a repeatable origami design with 3 pairs of SQ H1 and an example of nick, and the lower view illustrates comparison of migration during a repeatable origami process by gel electrophoresis using 0.8 wt.% of agarose gel stained with EtBr, and representative AFM images were measured after gel extraction to confirm the morphology of the unfolded (box of “U”) and folded (box of “F”) states in each band, wherein a scale-bar indicates 100 nm. (c) shows kinetics of origami-dependent fluorescence intensity, and the upper view is the expected luminous state in the initial folded state and unfolded state. Herein, two quenchers (quencher Q: EBQ) and a fluorophore (reporter R: Cy3) were arranged at a terminal of a protruding strand at the inner vertex of SQ, wherein bold arrows indicate the process of adding glue and releaser strands, respectively. The lower view shows kinetics of fluorescence intensity at 563 nm wavelength for 140 min, wherein light colored graph denotes 3 pairs of SQ H1, dark colored graph denotes 3 pairs of SQ H2, and a solid line was fit to a kinetic model derived therefrom. SQ H1 kₒₙ = 4.32 × 10⁴ 1/(M • s) , SQ H2 kₒₙ = 4.25 × 10⁴ 1/(M • s) , wherein a scale-bar indicates 100 nm. (d) shows mountain and valley folds, wherein the upper view is a diagram depicting 3 pairs of SQ H1 crease, mountain and valley folds, and the lower view shows kinetics of normalized fluorescence intensity for 15 minutes after convergence is reached. In (c) and (d) , the fluorescence intensity was normalized to compensate for dilution effects;

FIG. 17 is representative AFM images of orthogonal origami, wherein a scale-bar indicates 1 µm;

FIG. 18 is representative AFM images and illustrates results of gel electrophoresis for repeatable origami. Herein, the gel electrophoresis was performed with 0.8 wt.% of agarose gel stained with EtBr for 90 minutes at 75 V, which includes: Step 1 of SQ H1 (3 pairs, nick); Step 2 of adding 2 times the glue strands to the number of folding line handle pairs; Step 3 of adding 10 times the releaser strands to the added glue strands; Step 4 of adding 20 times the glue strands to the number of crease handle pairs (since there are 18 times the unbound releaser strands remaining in the glue strands in Step 3, the final concentration of the glue strands in Step 4 is twice that of the crease handle pair); and Step 5 of adding 10 times the releaser strands to the added glue strands. Herein, each AFM measurement for the band (boxes of “1”, “2”) was performed after gel extraction and filtration, and at least three AFM images were obtained to estimate the origami yield, wherein a scale-bar indicates 1 µm;

FIG. 19 is a graph illustrating results of dynamic analysis;

FIG. 20 is representative AFM images for origami-dependent fluorescence intensities, which includes: Phase I of an initial state; Phase II of adding the glue strands, and Phase III of adding the releaser strands. All AFM images were acquired after the fluorescence intensity of each phase was completely converged. Colored dots at the top of the images indicate the status of two reporters (Cy3), wherein dark dot denotes emission and light dot denotes evanishment, and a scale-bar indicates 1 µm;

FIG. 21 is representative AFM images of the mountain and valley-shaped folds of SQ H1, wherein a scale-bar indicates 1 µm; and

FIG. 22 illustrates an example of a conventional DNA origami structure.

### DETAILED DESCRIPTION

Hereinafter, the present invention will be described in detail.

The present invention relates to a method for preparing a nucleic acid structure, which includes:
a first step of binding at least one pair of first single-stranded nucleic acids protruding in the same direction to portions symmetric with respect to at least one crease line segment, respectively, in a nucleic acid wireframe having a plurality of line segments;
a second step of treating the nucleic acid wireframe with a second single-stranded nucleic acid which includes a first portion including a complementary sequence to the pair of first single-stranded nucleic acids and a second portion including a non-complementary sequence to the pair of first single-stranded nucleic acids at a terminal of the first portion, thus to bind the pair of first single-stranded nucleic acids to the second single-stranded nucleic acid.

The nucleic acid wireframe refers to a skeleton structure consisting of a wire made of nucleic acid. Each wire may be a single strand or double strand depending on the used nucleic acid, or may include the corresponding strand in multiple bundles.

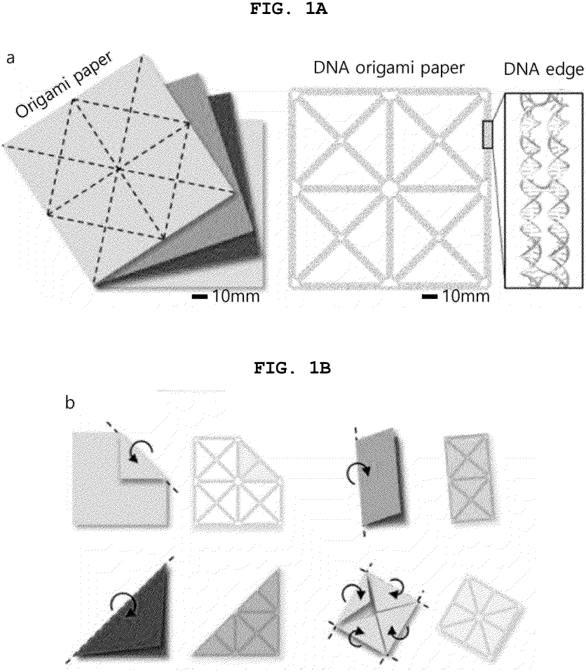

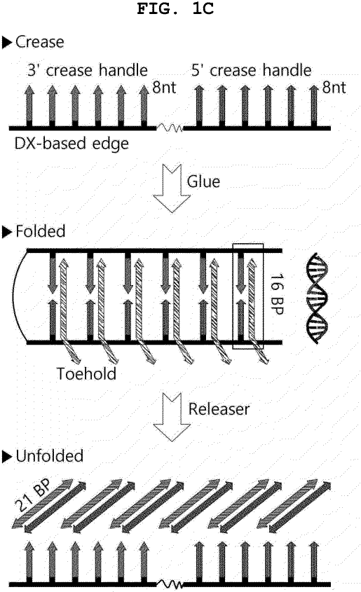

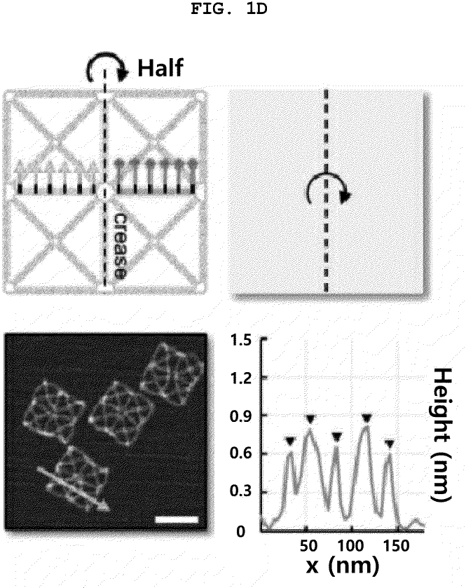

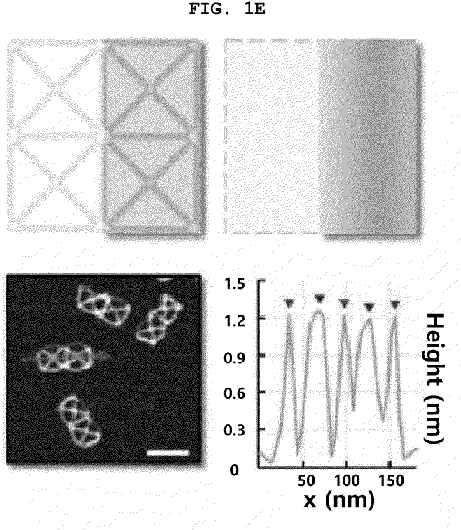

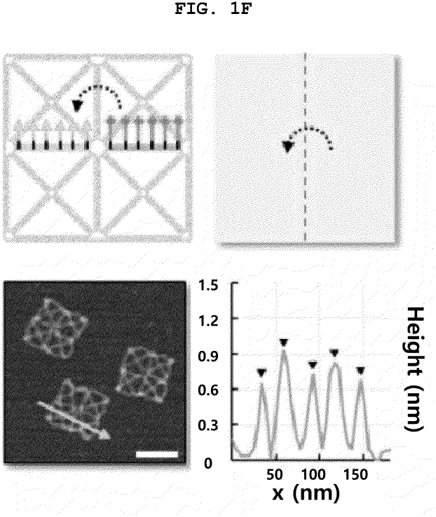

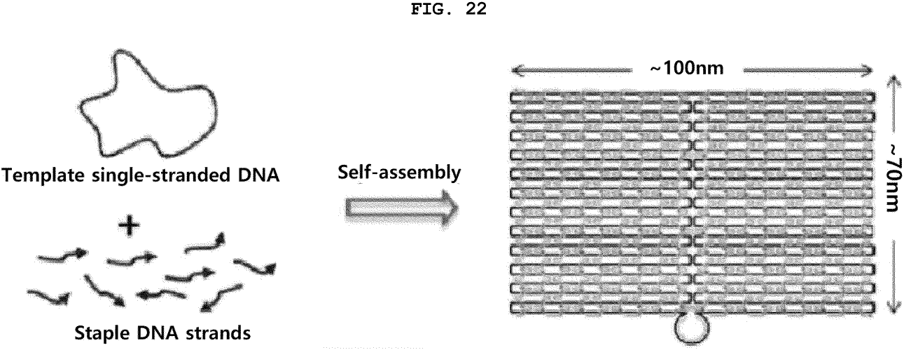

FIG. 1A illustrates an example of a nucleic acid wireframe (DNA origami wireframe), and FIG. 22 illustrates an example of a conventional DNA origami structure. Unlike the conventional DNA origami structure of FIG. 22, in which nucleic acid strands or bundles densely fill the plane, the nucleic acid wireframe consists only of wires.

The nucleic acid may be DNA or RNA, and the nucleic acid wireframe may be prepared through DNA origami or RNA origami technique. The DNA or RNA origami technique refers to a technique for preparing a desired structure by folding a long nucleic acid strand having about 7,000 to 8,000 bases into several tens to hundreds of short nucleic acid strands and immobilizing the same. Specifically, nucleic acid strands of specific pre-programmed sequences are synthesized using the Watson-Crick binding law to prepare a structure having a desired shape, then the nucleic acid self-assembles with other nucleic acids having a complementary sequence thereto to form a double-stranded nucleic acid. Using the same principle, two double-stranded nucleic acids can be linked in parallel through a binding site (folded portion), which is called “Holliday junction”. When linking a plurality of double-stranded nucleic acids in this way, it is possible to produce a nucleic acid nanostructure having a specific shape on a two-dimensional plane, and when extending the same principle onto a space, a three-dimensional structure having a specific lattice structure is prepared.

In the preparation method of the present invention, the nucleic acid wireframe has a lower internal rigidity than a structure including a surface or a thickness due to the flexibility thereof, such that more complete folding may be implemented.

The nucleic acid wireframe includes a plurality of line segments. As used herein, the line segment refers to a line that can be connected on a wireframe from one end to the other end of the wireframe, from one end to an intersection with another wireframe, or from one intersection to another intersection, etc.

The nucleic acid wireframe includes a frame including a two-dimensional mesh structure which has a figure of a cross shape or a triangular, quadrangular, hexagonal shape as a unit structure, which is formed by a plurality of nucleic acid line segments, and for example, may be a mesh structure which has a basic figure such as a right angled isosceles triangle, an equilateral triangle, and a square, etc., as the unit structure. The wireframe having such the mesh structure is advantageous in securing the line segment capable of being folded in various directions. The nucleic acid wireframe may be folded based on at least one line segment.

The preparation method of the present invention includes the first step of binding a pair of first single-stranded nucleic acids protruding in the same direction to portions symmetric with respect to the crease line segments, respectively, in the nucleic acid wireframe having a plurality of line segments.

The crease line segment may be a line segment that should be folded for preparing a structure intended or targeted by a user, and may be at least one of a plurality of line segments.

The first single-stranded nucleic acid is a nucleic acid which is complementary bound to a second single-stranded nucleic acid to be described below so as to allow the nucleic acid wireframe to be folded.

The first single-stranded nucleic acids are respectively bound to another first single-stranded nucleic acid protruding in the same direction at portions symmetric with respect to the crease line segment in the nucleic acid wireframe. Thereby, when folding the nucleic acid wireframe with respect to the line segment, the respective first single-stranded nucleic acids may be adjacent to or close to each other.

FIG. 1C schematically illustrates a mechanism by which the nucleic acid wireframe is folded. In FIG. 1C, a crease handle corresponds to the “first single-stranded nucleic acid”. A portion described as a zigzag between an edge and an edge in the crease handles corresponds to the “crease line”. When folding the nucleic acid wireframe based on the crease line, the respective first single-stranded nucleic acids are adjacent to or close to each other.

Then, when treating it with the “second single-stranded nucleic acid” described below, the pair of first single-stranded nucleic acids and the second single-stranded nucleic acid are complementary to each other, such that the folded structure is immobilized.

The pair of first single-stranded nucleic acids may have the same or different sequences, and may consist of two single-stranded nucleic acids having different sequences.

The pair of first single-stranded nucleic acids may be DNA or RNA, and types thereof may be selected according to the nucleic acid of the nucleic acid wireframe or the nucleic acid of the second single-stranded nucleic acid.

Folding strength, angle, etc. may be controlled by a length of the first single-stranded nucleic acid. The first single-stranded nucleic acid may have a length of, for example, 5 to 20 bp, 5 to 15 bp, 5 to 10 bp, respectively, but it is not limited thereto.

Binding the first single-stranded nucleic acids protruding in the same direction to the portions symmetric with respect to the crease line segments, respectively, includes the case in which the respective first single-stranded nucleic acids protrude into the same space in two spaces divided on the basis of a plane including the crease line segment and the portion symmetric with respect to the crease line segment.

The first single-stranded nucleic acids may be bound orthogonal to the portions symmetric with respect to the crease line segments in the nucleic acid wireframe in the same direction. When binding the nucleic acids orthogonal to the portions, ends of the respective first single-stranded nucleic acids are not misaligned in the folded state, such that binding to the second single-stranded nucleic acid in the second step may be easily performed.

When there is a plurality of crease line segments, binding of the first single-stranded nucleic acids may be performed for each crease line segment. For example, when there are two crease line segments, a pair of first single-stranded nucleic acids protruding in the same direction are respectively bound to portions symmetric with respect to one of the line segments, and similarly, another pair of the first single-stranded nucleic acids are respectively bound portions symmetric with respect to the remaining line segment. The wireframe may be folded by complementary binding of the first single-stranded nucleic acid and the second single-stranded nucleic acid to be described below. Therefore, when there is a plurality of crease line segments, sequences of the first single-stranded nucleic acids used for each crease line segment may be the same or different from each other.

A plurality of first single-stranded nucleic acids protruding in the same direction at the portions symmetric with respect to the crease line segments may be used for each portion. In this case, a plurality of second single-stranded nucleic acids to be described below are also used for each portion, and the plurality of first single-stranded nucleic acids and the plurality of second single-stranded nucleic acids are respectively bound to each other, such that the folded structure may be more strongly immobilized.

Thereafter, the nucleic acid wireframe is treated with a second single-stranded nucleic acid which includes a first portion including a complementary sequence to the pair of first single-stranded nucleic acids and a second portion including a non-complementary sequence to the pair of first single-stranded nucleic acids at a terminal of the first portion, thus to bind the pair of first single-stranded nucleic acids to the second single-stranded nucleic acid (second step).

The second single-stranded nucleic acid includes the first portion including a complementary sequence to each of the first single-stranded nucleic acids.

Since two first single-stranded nucleic acids and one second single-stranded nucleic acid may be bound to each other, the first portion of the second single-stranded nucleic acid includes complementary sequences to each of the two first single-stranded nucleic acids. The complementary sequence includes those that are partially or completely complementary to each of the first single-stranded nucleic acids, and includes those that include contiguous or discontinuous complementary sequences to each of the first single-stranded nucleic acids. Thereby, the folded structure may be immobilized by binding the second single-stranded nucleic acid to the first single-stranded nucleic acid.

The second single-stranded nucleic acid includes the second portion including a non-complementary sequence to the first single-stranded nucleic acid at the terminal of the first portion. Thereby, as illustrated in FIG. 1C, the second portion may not bind to the first single-stranded nucleic acid (toehold portion), which may be used for binding to a third single-stranded nucleic acid to be described below.

The second single-stranded nucleic acid may be DNA or RNA, and types thereof may be selected according to the nucleic acid of the nucleic acid wireframe, the nucleic acid of the first single-stranded nucleic acid, or the nucleic acid of the third single-stranded nucleic acid.

The second single-stranded nucleic acid may have a length of 10 to 40 bp, 10 to 30 bp, 10 to 20 bp, etc., but it is not limited thereto. The first portion and the second portion may be appropriately selected within the above range of the length, which may be selected in consideration of a binding force to the first single-stranded nucleic acid and a binding force to the third single-stranded nucleic acid. For example, the first portion may be 95%, 90%, 85%, 80%, 75%, 70%, 65%, 60%, 55%, 50% of a total base, but it is not limited thereto.

When there is a plurality of crease line segments and binding of the first single-stranded nucleic acids is performed for each crease line segment, the second single-stranded nucleic acids may also be treated so that they are bound to the first single-stranded nucleic acids by matching with the number of the crease line segments. In addition, when a plurality of first single-stranded nucleic acids protruding in the same direction are used for each portion at the portions symmetric with respect to the crease line segments, the second single-stranded nucleic acids may also be treated so that they are bound to the first single-stranded nucleic acids by matching with the number of the symmetrical portions.

The preparation method of the present invention may include a third step of adding a third single-stranded nucleic acid which includes a complementary sequence to the first portion and the second portion, thus to bind the second single-stranded nucleic acid to the third single-stranded nucleic acid so as to be separated from the first single-stranded nucleic acid.

As illustrated in FIG. 1C, the second single-stranded nucleic acid includes the second (toehold) portion including a non-complementary sequence to the first single-stranded nucleic acid, but the third single-stranded nucleic acid may include the complementary sequence to the first portion and the second portion, thus to be bound to the second single-stranded nucleic acid. In this case, the second single-stranded nucleic acid is separated from the first single-stranded nucleic acid, and then the folded structure may be unfolded.

The third single-stranded nucleic acid may have a stronger binding force to the second single-stranded nucleic acid to be bound than that of the two first single-stranded nucleic acids bound to the second single-stranded nucleic acid. To this end, the third single-stranded nucleic acid may be more complementary to the second single-stranded nucleic acid than the two first single-stranded nucleic acids.

The complementary sequence includes those that are partially or fully complementary to each of the first and second portions, and includes those that include contiguous or discontinuous complementary sequences to each of the first and second portions.

The third single-stranded nucleic acid may be DNA or RNA, and types thereof may be selected according to the nucleic acid of the nucleic acid wireframe, the nucleic acid of the first single-stranded nucleic acid or the nucleic acid of the second single-stranded nucleic acid.

The third single-stranded nucleic acid may have a length which is determined depending on the length of the second single-stranded nucleic acid, may have a length difference from the second single-stranded nucleic acid of less than 10 bp, less than 8 bp, less than 5 bp, or less than 3 bp, for example, and may have the same length as the second single-stranded nucleic acid.

Since the binding reaction of the single-stranded nucleic acids is a reversible reaction, it is possible to repeat the process of unfolding and refolding the folded structure using the same in the preparation method of the present invention.

Accordingly, by repeating the process of folding and unfolding in one nucleic acid wireframe based on different folding lines through the above preparation method, it is possible to repeatedly transform the one nucleic acid wireframe into various shapes.

The preparation method of the present invention may include binding a fluorescent material and a quencher to both sides of the nucleic acid wireframe so as to be symmetric with respect to the crease line segment, respectively, before the second step.

The quencher refers to a molecule that acts on a molecule present in an excited state like the fluorescent material, to lose energy by energy transfer, electron transfer, or other chemical process so as to return it to the ground state.

As the quencher, materials known to have quenching performance in the art may be used without limitation, and for example, graphene oxide may be used.

The fluorescent material is not particularly limited as long as it is a material capable of reducing a fluorescence signal by the action of the quencher, and may include, for example, Cy3, Cy5, thiourea (FTH), 7-acetoxycoumarin-3-yl, fluorescein-5-yl, fluorescein-6-yl, 2′,7′-dichlorofluorescein-5-yl, 2′, 7′-dichlorofluoresin-6-yl, dihydrotetramethylrosamine-4-yl, tetramethylrhodamine-5-yl, tetramethylrhodamine-6-yl, 4,4-difluoro-5,7-dimethyl-4-bora-3a,4a-diaza-s-indacene-3-ethyl or 4,4-difluoro-5,7-diphenyl-4-bora-3a,4a-diaza-s-indacene-3-ethyl, poly L-lysine-fluorescein isothiocyanate (FITC), rhodamine-B-isothiocyanate (RITC), phycoerythrin (PE) or rhodamine. The fluorescent material and the quencher are respectively bound to both sides of the nucleic acid wireframe so as to be symmetric with respect to the crease line segment, and when the line segment is folded, a distance between the quencher and the fluorescent material changes, such that they may be adjacent to or close to each other.

Fluorescence intensity of the fluorescent material may change according to the change in the distance between the quencher and the fluorescent material. Conversely, information on the change in the distance between the quencher and the fluorescent material, that is, the change in the structure of the nucleic acid wireframe in which the quencher and the fluorescent material are bound, may be obtained through the change in the fluorescence intensity.

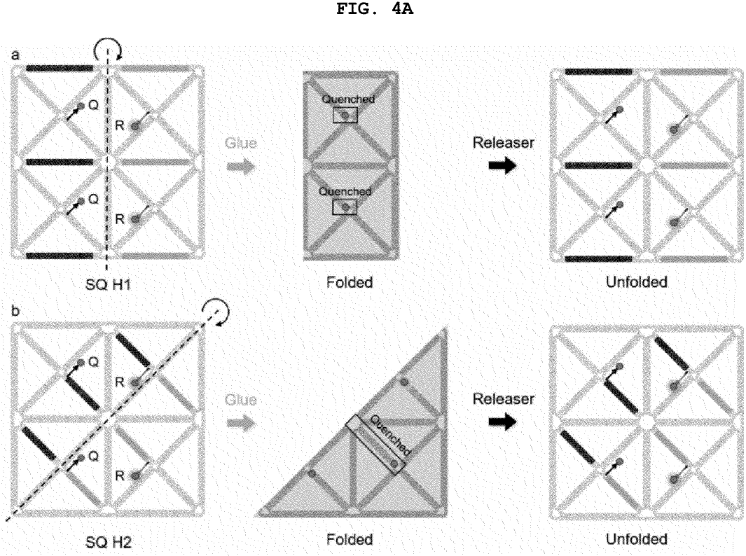

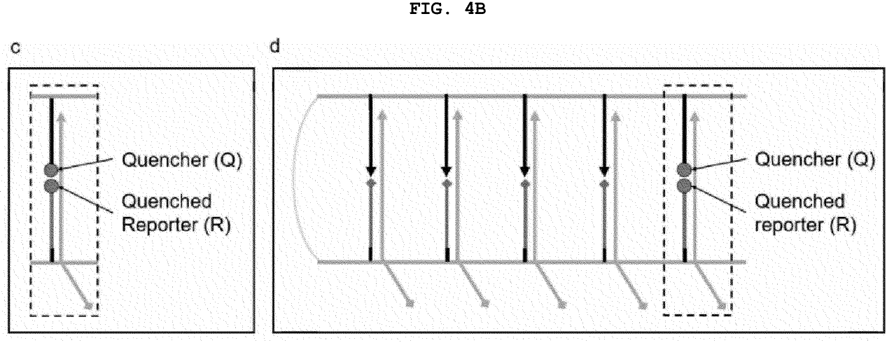

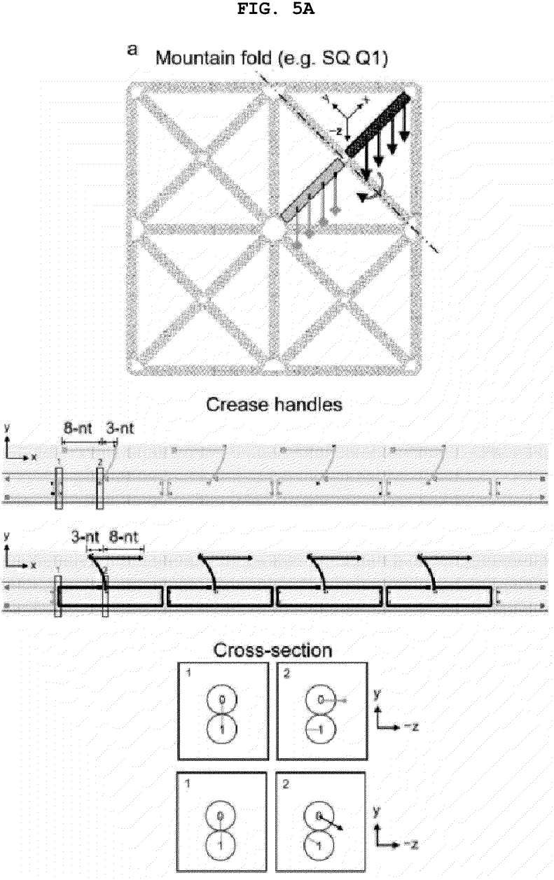

For example, FIG. 4A and FIG. 4B illustrate an appearance in which the fluorescent material and the quencher are bound to both sides of the nucleic acid wireframe so as to be symmetric with respect to the line segment folded before and after the second step (Glue in FIG. 4A). Since positions of the fluorescent material and the quencher symmetric to the crease line segment before the second step are adjacent to each other after the second step (Folded in FIG. 4A), the fluorescence intensity may be weaker than before the second step.

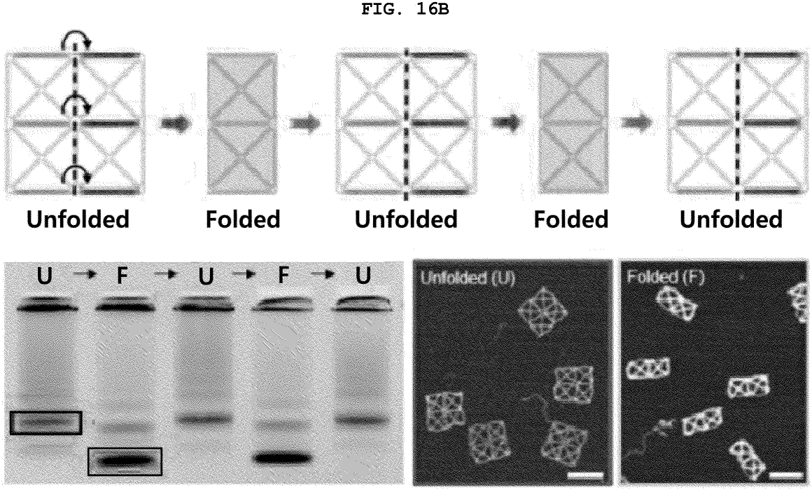

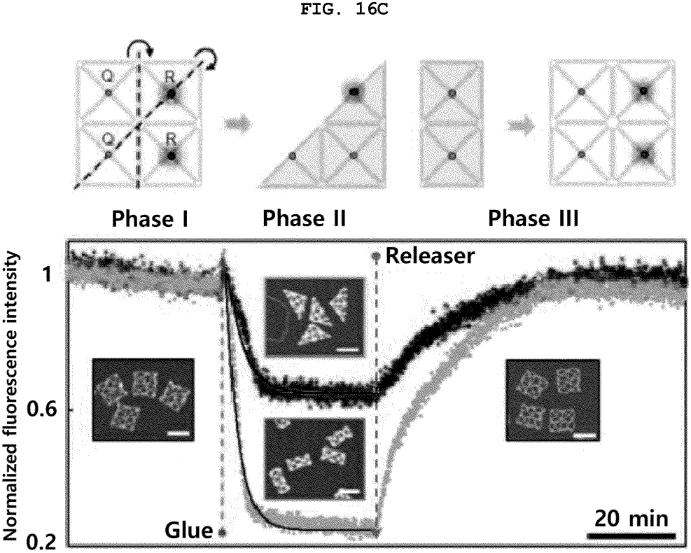

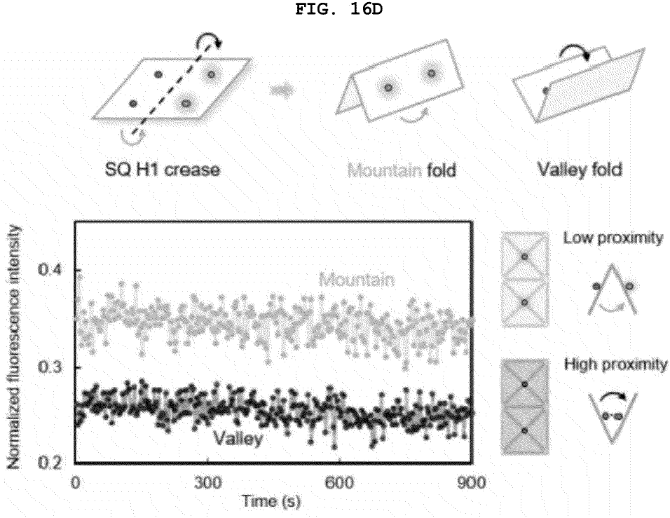

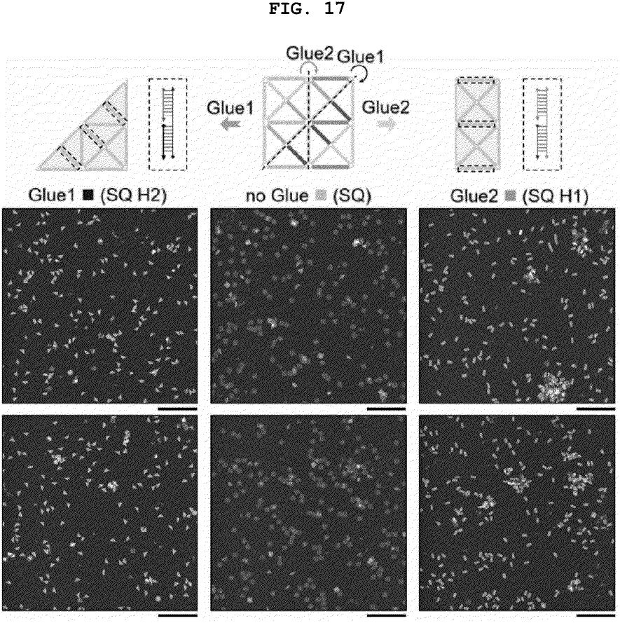

FIG. 16D is a schematic diagram illustrating an appearance of folding outward (Mountain fold) and inward folding (Valley fold) a surface where quenchers and fluorescent materials are bound to the nucleic acid wireframe, when the fluorescent material and the quencher are bound so as to be symmetric with respect to the crease line segment (dotted line). Since a distance between the fluorescent material and the quencher in the inward folded state is closer (high proximity), a lower fluorescence intensity than the case of being folded outward (low proximity) is exhibited.

The preparation method of the present invention includes the third step of adding a third single-stranded nucleic acid which includes a complementary sequence to the first portion and the second portion, thus to bind the second single-stranded nucleic acid to the third single-stranded nucleic acid so as to be separated from the first single-stranded nucleic acid. In the third step, the folded structure due to the second step may be unfolded, and the fluorescence intensity may change as the distance between the fluorescent material and the quencher is back its original state.

The quencher and the fluorescent material may be bound to terminals of each of the first single-stranded nucleic acids bound in the same direction to the portions symmetric with respect to the crease line segment. When the terminals of the respective first single-stranded nucleic acids are bound orthogonal to the symmetrical portions in the same direction (c of FIG. 4B), the quencher and the fluorescent material are not misaligned in the folded state, such that the change in the fluorescence intensity of the fluorescent material due to the quencher may be easily observed.

When using the preparation method of the present invention, mobility of the nucleic acid wireframe may be controlled by selectively folding or unfolding the nucleic acid wireframe in a biological material according to the treatment of the second single-stranded nucleic acid or the third single-stranded nucleic acid.

Furthermore, by loading a material on the nucleic acid wireframe and wrapping or exposing the loaded material to an outside, the inventive method has an effect of ergonomically designing transferability of the loaded material, and thereby it may be utilized for drug delivery.

When binding the fluorescent material and the quencher as described above, information on the folded and unfolded states may be obtained, such that the migration of the nucleic acid wireframe as described above, the migration of the loaded material, and whether the loaded material is exposed may be determined.

As used herein, designing the crease line along the line segment inside the nucleic acid wireframe structure as described above may be used interchangeably with the term ‘crease design’, and the designed crease line segment may be used interchangeably with the term ‘crease pattern’.

In the present disclosure, each of the first single strands may be used interchangeably with the terms ‘protruding strand’ and ‘crease handle’.

In the present disclosure, the second single strand may be used interchangeably with the term ‘glue strand’.

In the present disclosure, the ‘second portion’ may be used interchangeably with the term ‘toehold’.

In the present disclosure, the third single strand may be used interchangeably with the term ‘releaser strand’.

In addition, the present invention relates to a method for detecting a target nucleic acid, which includes a first step of binding at least a pair of first single-stranded nucleic acids protruding in the same direction to portions symmetric with respect to at least one crease line segment, respectively, in a nucleic acid wireframe having a plurality of line segments; and

a second step of treating the nucleic acid wireframe with a sample suspected of containing a target nucleic acid which includes a first portion including a complementary sequence to the pair of first single-stranded nucleic acids and a second portion including a non-complementary sequence to the pair of first single-stranded nucleic acids at a terminal of the first portion, thus to confirm whether the wireframe is folded at the line segment.

The detection includes all action (e.g., fluorescence change, absorption change, etc.) for confirming the presence of a specific material through a selective reaction for the specific material.

The target nucleic acid is a material or a substance bound thereto, which is used for understanding the presence or absence. The material is not particularly limited as long as it can include the nucleic acid, and may be, for example, a biomarker or target molecule for diagnosing a disease.

The target nucleic acid includes the first portion and the second portion, and the description of these portions is overlapped with the description of the second single-stranded nucleic acid of the above-described preparation method, and therefore will not be described.

When treating the nucleic acid wireframe with the sample suspected of containing the target nucleic acid, if the target nucleic acid including the first portion and the second portion is included in the sample, since the folded structure is immobilized by binding the first portion to each of the first single-stranded nucleic acids protruding in the same direction at the portions symmetric with respect to the crease line segment, it is possible to understand that the target nucleic acid is present by confirming whether the line segment is folded.

When the line segment is folded, it is possible to determine that the target nucleic acid corresponding to the pair of first single-stranded nucleic acids which are bound thereto corresponding to the folded line segment is present in the sample.

For detection of a target nucleic acid, the first single-stranded nucleic acid may be designed and used to satisfy homology and non-homology with the first and second portions of the target nucleic acid. There may be a plurality of crease line segments. In this case, binding of the first single-stranded nucleic acids may be performed for each crease line segment.

The detection method of the present invention may be used to detect one target nucleic acid or a plurality of different target nucleic acids.

When detecting one target nucleic acid, if there is one crease line segment, it is possible to confirm the presence or absence of the target nucleic acid by determining whether the line segment is folded. When there is a plurality of crease line segments, since more target nucleic acids are present in the sample, more line segments can be folded, such that it is possible to not only detect the target nucleic acid but also confirm the relative number or concentration thereof.

When detecting a plurality of different nucleic acids, if there is a plurality of crease line segments, the first single-stranded nucleic acid capable of binding to different target nucleic acids may be bound to each crease line segment. In this case, if the plurality of different target nucleic acids is present, a plurality of line segments is folded, such that the presence or absence of the plurality of different target nucleic acids may be confirmed by the number of the crease line segments.

The detection method may include the step of binding a fluorescent material and a quencher to both sides of the wireframe so as to be symmetric with respect to the crease line segment, respectively, before the second step.

The description of the step of binding the fluorescent material and the quencher to the wireframe is overlapped with the description in the above-described preparation method, and therefore will not be described.

The second step of confirming whether the line segment of the wireframe is folded may include measuring a change in the fluorescence intensity of the fluorescent material. When the fluorescence intensity of the fluorescent material is decreased after treating the sample, it is possible to determine that the line segment is folded.

In addition, an amount of the target nucleic acid in the sample may be quantified based on a degree of a decrease in the fluorescence intensity.

For example, when a plurality of pairs of first single-stranded nucleic acids symmetric with respect to the crease line segment are present, more complete folding is achieved in proportion to the amount of the target nucleic acid present in the sample, and thereby the fluorescence intensity may be greatly reduced. Alternatively, when there is a plurality of crease line segments, the more the target nucleic acids are present in the sample, the more the line segments are folded, such that the fluorescence intensity may be reduced in proportion thereto.

Further, the present invention relates to a nucleic acid structure including: a nucleic acid wireframe having a plurality of line segments; and at least a pair of first single-stranded nucleic acids which protrude in the same direction and are bound to portions symmetric with respect to at least one crease line segment in the same direction in the nucleic acid wireframe, wherein among a first portion and a second portion adjacent to each other in a target nucleic acid sequence, the pair of first single-stranded nucleic acids have a complementary sequence to at least a part of the first portion and a non-complementary sequence to the second portion.

The nucleic acid wireframe may include a DNA origami wireframe, and the nucleic acid structure may include a third single-stranded nucleic acid including a complementary sequence to the first portion and the second portion.

The pair of first single-stranded nucleic acids may have the same or different sequences, and may consist of two single-stranded nucleic acids having different sequences.

The nucleic acid wireframe may include a DNA origami wireframe.

The nucleic acid structure may have a structure in which a fluorescent material and a quencher are bound respectively to both sides of the wireframe so as to be symmetric with respect to the crease line segment.

As in the method for preparing a nucleic acid structure described above, the structure may be a nucleic acid structure in which a pair of first single-stranded nucleic acids are bound symmetrically to a nucleic acid line segment that becomes a foldable line on the nucleic acid wireframe having a plurality of line segments, and each first single-stranded nucleic acid is bound to the second single-stranded nucleic acid, thus to be folded using the crease line segment as a folding line.

By repeating the process of folding and unfolding the structure based on different folding lines in the nucleic acid wireframe, it is possible to repeatedly transform one nucleic acid wireframe into various shapes. The respective terms, the principles and effects of the invention are overlapped with the description in the above-described preparation method, and therefore will not be described.

Hereinafter, the present invention will be described in detail with reference to examples.

### Example

### Experimental Method

### 1. Design and Synthesis of DNA Paper Wireframe

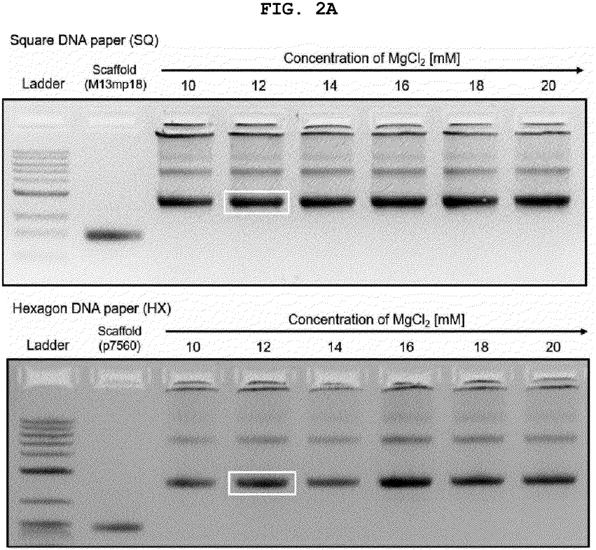

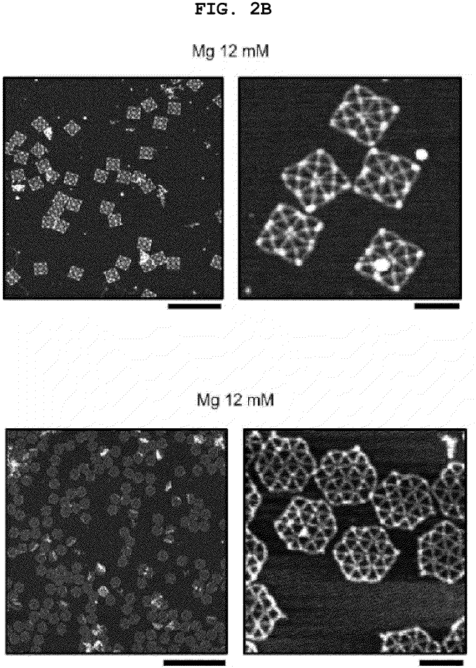

Square (SQ) and hexagonal (HX)-shaped wireframe DNA papers were designed using PERDIX. Designs of the vertex and corner staple have been edited to implement a mechanically foldable portion and a folding line. The specific configuration thereof is shown in FIG. 2A and FIG. 2B.

Two types of scaffolds of M13mp18 (New England Biolabs) and p7560 (IDT) were used to design SQ and HX nanostructures, respectively. All staples were ordered on a 50 nmole scale, and initially diluted to 100 pmole/µL (BioRP purification, Bioneer Corporation). The final concentrations of the mixture were set to be 20 nM of scaffold, 100 nM of each staple, 1 × TAE buffer (with 40 mM Tris-acetate and 1 mM EDTA, Sigma-Aldrich) and 12 mM MgCl₂. A PCR annealing process was performed for a total of 42 hours using a thermal cycler (T100, Bio-Rad) . The mixture was heated to 80° C. at a rate of 1° C./s, cooled from 80° C. to 65° C. for 1 hour (1° C. per 4 minutes), then cooled from 65° C. to 25° C. for 40 hours (1° C. per hour), and maintained at 4° C.

### 2. Ultrafiltration

The synthesized samples were purified with a 50 kDa molecular weight cutoff filter to remove excess staples. The filter was moistened with 12 mM MgCl₂ buffer in an amount of 500 µL, followed by rotating at 5,000 rcf (relative centrifugal force) for 8 minutes at 20° C. The filtrate was discarded, and the samples and buffer were sequentially poured into a filter tube, followed by centrifugation at 5,000 rcf for 8 minutes at 20° C. The above buffer exchange and filtration were repeated twice. Finally, the purified samples were collected by inverting the filter and rotating the tube at 10,000 rcf for 3 minutes at 20° C. All concentrations of the purified samples were measured by DNA absorbance measurements at 230 nm, 260 nm and 280 nm using a NanoDrop, and the specific procedures are described in Table 1 below.

TABLE 1

Effective concentration of DNA wireframe nanostructure

| Strand type | Pairing type | Square DNA paper (SQ) | Hexagon DNA paper (HX) |
|---|---|---|---|
| ssDNA [nt] | Unpaired scaffold | 42 | 86 |
| | Unpaired staple | 324 | 456 |
| | Scaffold loop | 63 | 166 |
| dsDNA [bp] | Base pair | 7144 | 7308 |
| Absorption coefficient | | 49.04 | 48.50 |
| Molecular weight | | 4,469,632 | 4,653,978 |

In Table 1, the concentrations of each DNA wireframe nanostructure were estimated in consideration of the number of single-stranded DNAs (ssDNAs) and double-stranded DNAs (dsDNAs) in the nanostructure. The total number of unpaired scaffolds, staples and base pairs might be obtained from PERDIX, and scaffold loops were intentionally added to adjust the scaffold lengths of p7249 for SQ and p7560 for HX. The absorption coefficient was calculated by performing linear interpolation with a value of 33 for the ssDNA and 50 for the dsDNA. An approximate average of four types of nucleic acids was used to estimate the total molecular weight of the structure.

### 3. Gel Electrophoresis and Extraction

The samples were subjected to electrophoresis with 0.8% to 1.5% of agarose gel in an ice-filled water bath (i-Myrun, Cosmo Bio Co., Ltd.) for 90 minutes at 75 V bias voltage (up to 3.7 V/cm) . Running buffer consisted of 0.5 × TBE (45 mM Tris-borate and 1 mM EDTA, Sigma-Aldrich), 12 mM MgCl₂, and 0.5 µg/mL of ethidium bromide (EtBr, Noble Bioscience Inc.), and gel images were scanned with a laser. GelDoc XR+ device and Image Lab v5.1 program (Bio-Rad) were used. Thereafter, the samples subjected to electrophoresis were carefully extracted with a laser blade and scrambled several times inside a Freeze N Squeeze tube (Bio-Rad) using tweezers. The samples were extracted by freezing for 5 minutes at -27° C. and performing centrifugation for 5 minutes at 7,000 rcf and 20° C.

### 4. Measurement of Atomic Force Microscopy (AFM)

The samples were diluted with a buffer consisting of 20 mM MgCl₂, 40 mM Tris-acetate and 1 mM EDTA, and deposited on freshly cut mica (top grade V1 AFM Mica, Ted-Pella Inc.) for 5 minutes. A substrate was carefully washed three times with 200 µL of deionized water, and then thoroughly dried with an N₂ gun (<0.1 Kgf/cm²). NX10 (Park Systems) and PPP-NCHR probe (Nanosensors) having a spring constant of 42 N/m were used for the measurement. All images were taken in a non-contact mode using SmartScan software and flattened in linear and quadratic order using XEI 4.1.0 program (Park Systems). Thereafter, a height of the structure was measured using line profile analysis of the program.

### 5. FE Simulation

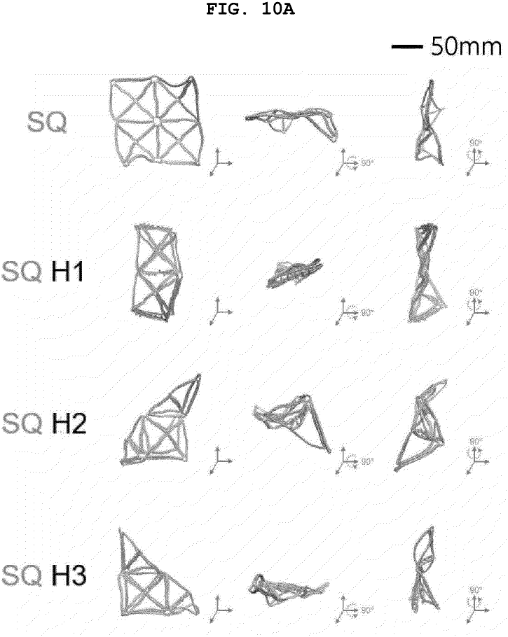

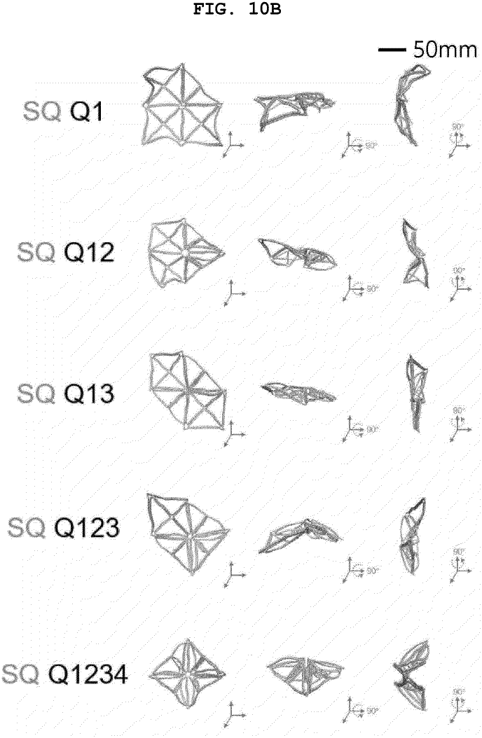

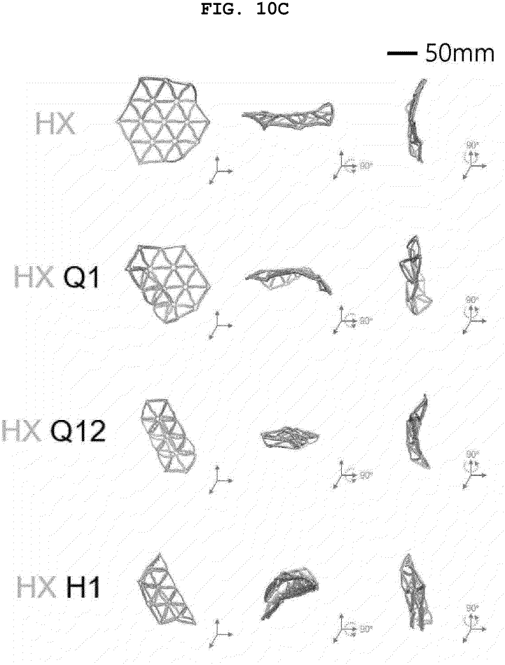

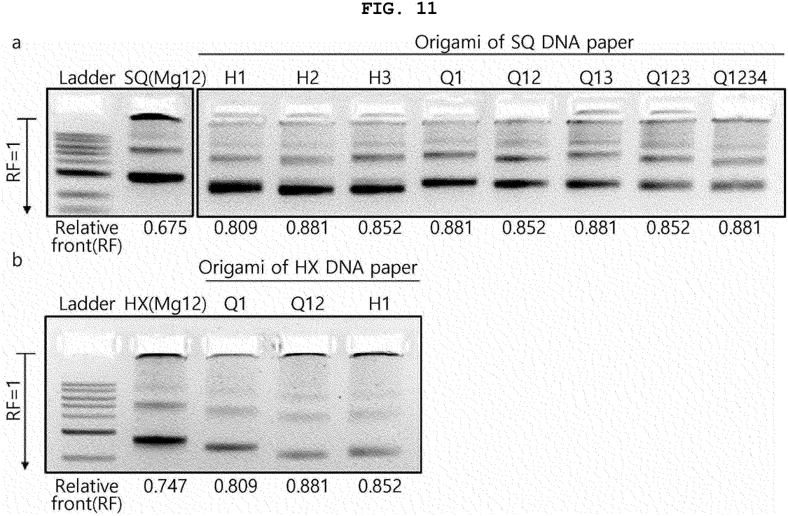

Equilibrium configuration and rigidity of wireframe DNA nanostructures were obtained using the finite element framework (SNUPI), to which division and rearrangement method are additionally applied. A general mode analysis of the structure was performed by allocating structural design and nucleotide sequence files, and introducing the recently searched properties of the gap. All shape results and parameters are shown in FIG. 10A to FIG. 10C and Table 2 below.

TABLE 2

Major parameters of finite element analysis

| | | | |
|---|---|---|---|
| Option | Abbreviation | Value | Unit |
| 1. Base-pair (BP) and crossover (CO) steps | | | |
| Coefficient function | BP_CF_IND | Order 1 | - |
| 2. Single-stranded DNA (ssDNA) | | | |
| Contour length per nucleotide | SS_LC1 | 0.67 | [nm/nt] |
| Persistence length | SS_LB | 0.74 | [nm] |
| Stretching rigidity when stretched | SS_EA_H | 710 | [pN] |
| Stretching rigidity when relaxed | SS_EA_L | 5 | [pN] |
| Coefficient function | SS_CF_IND | Order 1 | - |
| 3. Electrostatic interaction (ES) | | | |
| Temperature | ES_TEMP | 300 | [K] |
| Mg concentration | ES_MG | 20 | [mM] |
| Cutoff distance | ES_R_CUT | 20 | [nm] |
| Coefficient function | ES_CF_IND | Quadratic | - |

In Table 2 above, the division and rearrangement method were applied before the FE analysis, and values set in SNUPI were used for other parameters.

### 6. Yield Analysis

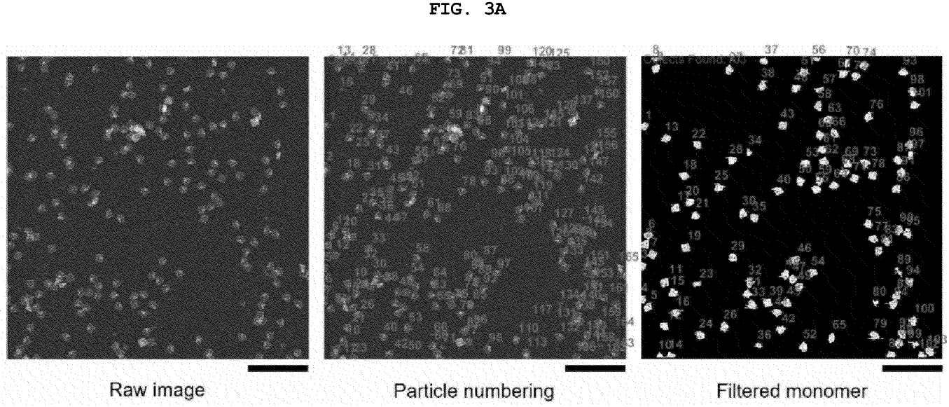

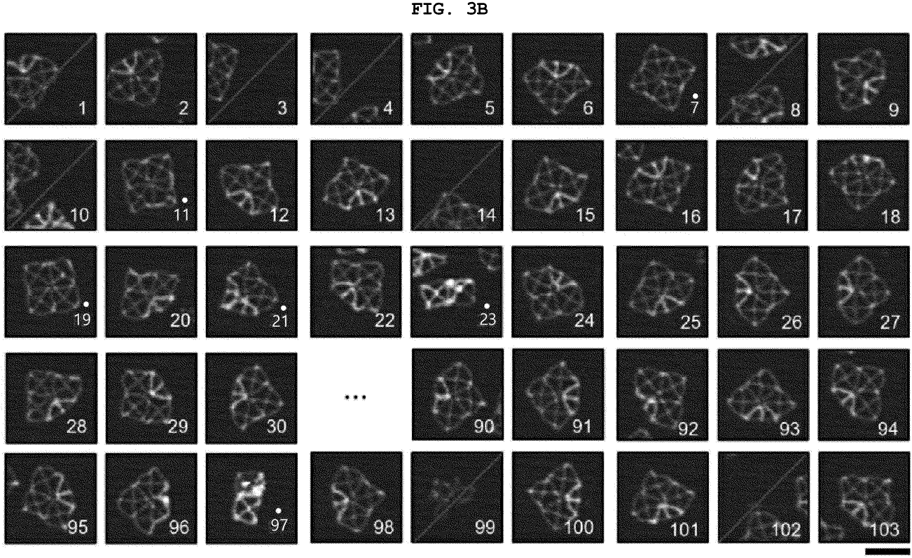

Information on the pixel size region and position of all particles in the AFM images was obtained by writing user-defined code in MATLAB’s Image Processing Toolbox. Thereafter, nanostructures agglomerated beyond the area criterion were systematically filtered, and individual images containing only one particle were automatically cut out and collected. Based on the collected individual images, the number of monomers with intended shape among the number of total monomers in the AFM images was counted to finally estimate each origami yield. Specific procedures and all results of the estimated yield are shown in FIG. 3A, FIG. 3B and Tables 3 to 8 below.

$$\text{Yield} \left(\%\right) = \frac{N u m b e r o f m o n o m e r w i t h int e n d e d s h a p e}{N u m b e r o f t o t a l m o n o m e r} \times 100 \%$$

TABLE 3

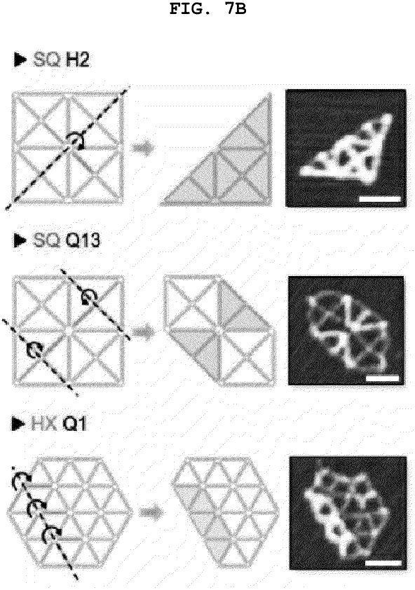

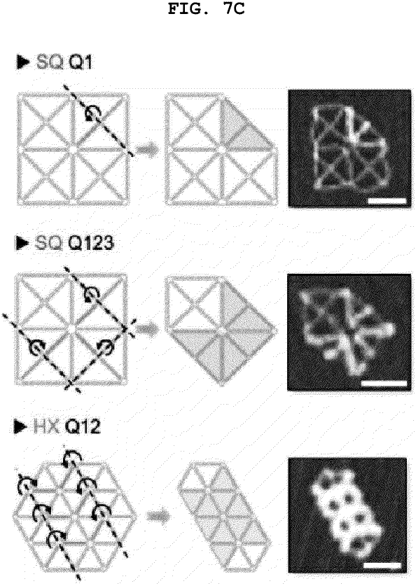

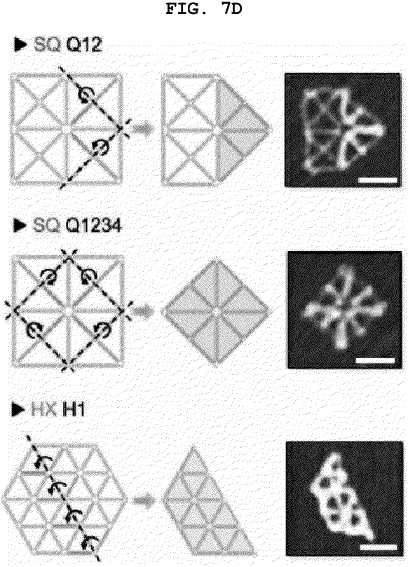

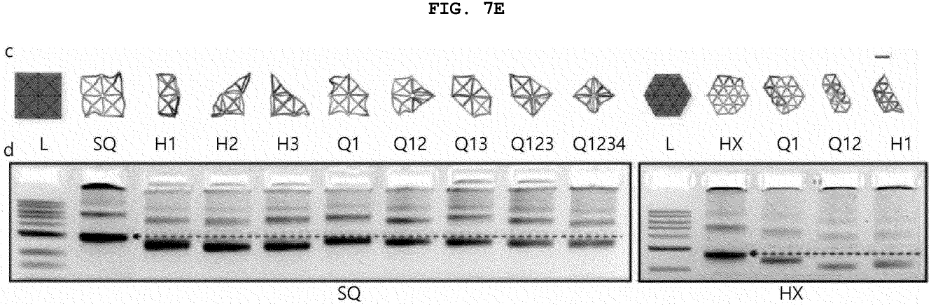

Single-folded origami

| Origami type | Number of Pair | Number of Gap (state) * FIGS. 7A to 7E | Number of DNA structures | | | Origami yield (%) | Standard deviation (%) |
|---|---|---|---|---|---|---|---|
| | | | Intended origami | Others | Total | | |
| SQ H1 | 1 (90-deg) | nick | 38 | 211 | 249 | 15.3 | - |
| | | 1-gap | 99 | 222 | 321 | 30.8 | - |
| | | 2-gap | 83 | 161 | 244 | 34.0 | - |
| | | 3-gap | 80 | 147 | 227 | 35.2 | - |
| | | 5-gap | 144 | 305 | 449 | 32.1 | 5.66 |
| | 1 | * nick | 343 | 165 | 508 | 67.5 | 2.73 |
| | | 1-gap | 404 | 120 | 524 | 77.1 | 2.48 |
| | | 2-gap | 385 | 183 | 568 | 67.8 | 2.78 |
| | | 3-gap | 457 | 137 | 594 | 76.9 | 1.09 |
| | | 5-gap | 485 | 112 | 597 | 81.2 | 1.79 |
| | 2 | nick | 508 | 61 | 569 | 89.3 | 4.51 |
| | | 1-gap | 340 | 49 | 389 | 87.4 | 1.33 |
| | | 2-gap | 466 | 68 | 534 | 87.3 | 1.62 |
| | | 3-gap | 391 | 60 | 451 | 86.7 | 1.40 |
| | | 5-gap | 429 | 50 | 479 | 89.6 | 3.21 |
| | 3 | nick | 91 | 14 | 105 | 86.7 | 3.69 |
| | | 1-gap | 140 | 19 | 159 | 88.1 | 5.38 |
| | | 2-gap | 143 | 11 | 154 | 92.9 | 1.90 |
| | | 3-gap | 267 | 34 | 301 | 88.7 | 1.89 |
| | | 5-gap | 176 | 13 | 189 | 93.1 | 1.25 |
| SQ H2 | 1 | * 4-nt unpaired | 119 | 192 | 311 | 38.3 | 4.13 |
| | 2 | 4-nt unpaired | 393 | 75 | 468 | 84.0 | 3.36 |
| | 3 | 4-nt unpaired (Glue×01 ) | 371 | 28 | 399 | 93.0 | 1.43 |
| | | 4-nt unpaired (Glue×02 ) | 302 | 25 | 327 | 92.4 | 2.78 |
| | | 4-nt unpaired (Glue×05 ) | 357 | 22 | 379 | 94.2 | 2.06 |
| | | 4-nt unpaired (Glue×10 ) | 445 | 29 | 474 | 93.9 | 1.33 |
| SQ Q1 | 1 | * nick | 126 | 47 | 173 | 72.8 | - |
| | | 3-gap | 189 | 64 | 253 | 74.7 | - |
| | 3 | nick | 291 | 40 | 331 | 87.9 | 2.56 |
| | | 1-gap | 512 | 67 | 579 | 88.4 | 0.97 |
| | | 5-gap | 468 | 97 | 565 | 82.8 | 0.87 |
| HX H1 | 2 | 5-nt unpaired | 140 | 172 | 312 | 44.9 | 2.81 |
| | 4 | * 5-nt unpaired | 175 | 73 | 248 | 70.6 | 5.78 |
| HX Q1 | 3 | * nick | 141 | 118 | 259 | 54.4 | 6.12 |
| | | 1-gap | 229 | 94 | 323 | 70.9 | 4.25 |
| | | 2-gap | 180 | 65 | 245 | 73.5 | 4.19 |
| | | 3-gap | 235 | 121 | 356 | 66.0 | 5.04 |
| | | 5-gap | 437 | 72 | 509 | 85.9 | 0.49 |

TABLE 4

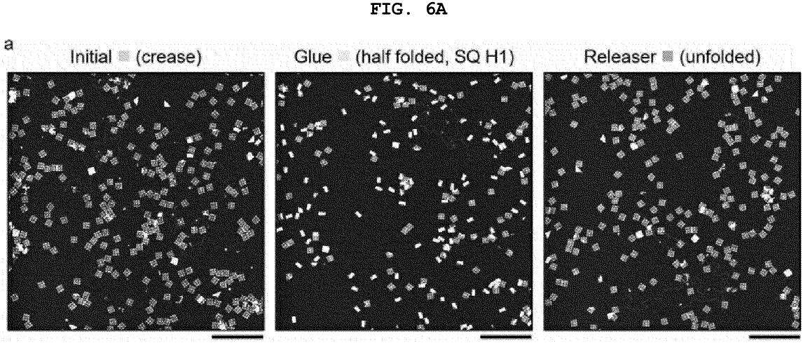

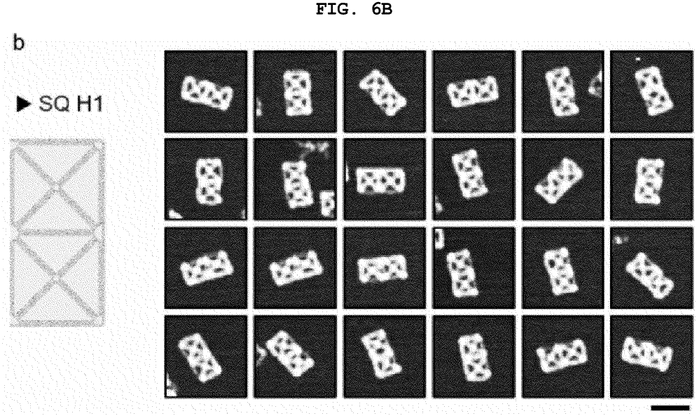

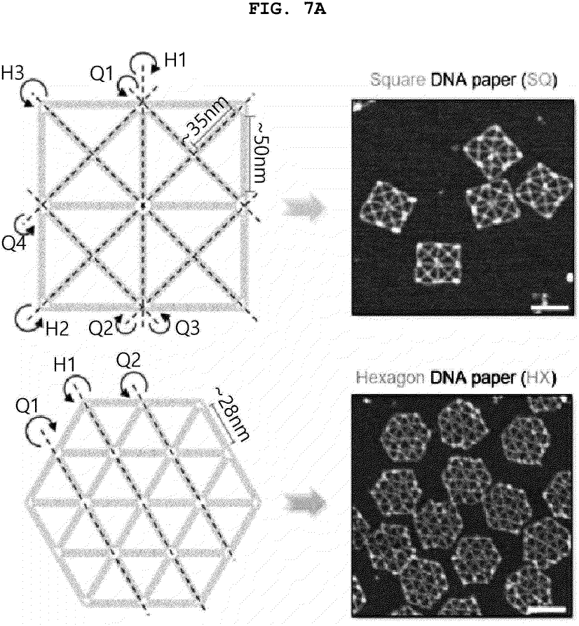

Multi-folded origami (SQ: 1-pair, nick, HX: 3-pair, nick) (FIG. 6A and FIG. 6B)

| Origami type | Number of DNA structures | | | | | | | | | | Origami yield (%) | Standard deviation (%) |
|---|---|---|---|---|---|---|---|---|---|---|---|---|
| | Q1 | Q12 | Q13 | Q123 | | Q1234 | | Others | | Total | | |
| SQ Q1 | 126 | - | - | | - | | - | | 47 | 173 | 72.8 | - |
| SQ Q12 | 122 | 150 | - | | - | | - | | 79 | 351 | 42.7 | 7.71 |
| SQ Q13 | 115 | - | 102 | | - | | - | | 69 | 286 | 35.7 | 5.94 |
| SQ Q123 | 38 | 57 | 27 | | 74 | | - | | 25 | 221 | 33.5 | 4.21 |
| SQ Q1234 | 15 | 21 | 22 | | 59 | | 25 | | 38 | 180 | 13.9 | - |
| HX Q1 | 141 | - | - | | - | | - | | 118 | 259 | 54.4 | 6.12 |
| HX Q12 | 55 | 40 | - | | - | | - | | 38 | 133 | 30.1 | 12.1 |

TABLE 5

Orthogonal origami (H1: 3-pair, nick, H2: 3-pair, 4-nt unpaired)

| Origami type | State | Number of DNA structures | | | | | | Yield(%) | Standard deviation (%) |
|---|---|---|---|---|---|---|---|---|---|
| | | Unfolded | H2 | H1 | Q1 | Others | Total | | |
| Orthogon al SQ (H1, H2) | no Glue | 242 | 7 | 7 | 28 | 9 | 293 | 82.6 | 1.72 |
| | Glue1 | 5 | 369 | 1 | 3 | 17 | 395 | 93.4 | 1.21 |
| | Glue2 | 1 | 4 | 453 | 2 | 11 | 471 | 96.2 | 1.24 |

TABLE 6

Repeatability (H1: 3-pair, nick, Gel extracted)

| Origami type | State | Number of DNA structures | | | | Yield (%) | Standard deviation (%) |
|---|---|---|---|---|---|---|---|
| | | Unfolded | H1 | Others | Total | | |
| SQ H1 | Unfolded | 128 | 7 | 23 | 158 | 81.0 | 5.69 |
| | Folded | 0 | 761 | 19 | 780 | 97.6 | 2.03 |

TABLE 7

Origami folded in mountain and valley shapes (H1: 3-pair, nick, internal temperature: 37° C.)

| Origami type | State | Number of DNA structures | | | | Yield (%) | Standard deviation (%) |
|---|---|---|---|---|---|---|---|
| | | H1 | Unfolded | Others | Total | | |
| SQ H1 | Mountain | 213 | 3 | 1 | 217 | 98.2 | 2.96 |
| | Valley | 276 | 0 | 5 | 281 | 98.2 | 1.07 |

TABLE 8

Origami-dependent fluorescence intensity (H1: 3-pair, nick, H2: 3-pair, 4-nt unpaired)

| Origami type | State | Number of DNA structures | | | | Yield (%) | Standard deviation (%) |
|---|---|---|---|---|---|---|---|
| | | Intended origami | Unfolded | Others | Total | | |
| SQ H1 | Initial | 17 (H1) | 265 | 60 | 342 | 77.5 | 2.93 |
| | Glue | 276 (H1) | 0 | 5 | 281 | 98.2 | 1.07 |
| | Releaser | 11 (H1) | 178 | 23 | 212 | 84.0 | 4.86 |
| SQ H2 | Initial | 23 (H2) | 397 | 75 | 495 | 80.2 | 2.67 |
| | Glue | 549 (H2) | 4 | 31 | 584 | 94.0 | 2.18 |
| | Releaser | 36 (H2) | 349 | 70 | 455 | 76.7 | 4.45 |

### 7. Folding and Unfolding of DNA Paper Wireframe

In general, the folding process was performed by adding glue strands at a concentration 10 times higher than that of the diluted nanostructures after filtration. The samples were stored at room temperature overnight. Folding was performed by adding releaser strands 10 times higher than the concentration of the glue strands. In order to activate Toehold-mediated DNA displacement, the samples were incubated for 1 hour at 37° C.

### 8. FRET Measurement

Vertex strands of a square DNA paper were modified with Cyanine 3 and a quencher (Bioneer) as shown in FIG. 4A and FIG. 4B. Before measurement, the samples were filtered and diluted to 20 nM, and a sample without fluorescent molecules was measured in advance for use as a blank to remove background noise. When excited at 546 nm wavelength, emission spectral analysis and kinetics at 563 nm wavelength were performed using a HORIBA Scientific Jobin Yvon Spectrofluorometer with Dual-FL software 3.7 and a 100 µL cuvette (Hellma) having a 10 × 2 mm optical path. All fluorescence intensity data were normalized to compensate for dilution effects.

### Experiment Result

### 1. Design of DNA Origami

A prerequisite for the effective transfer of macroscale origami technique to DNA nanotechnique is to obtain a layered platform flexible enough to fully fold and unfold the nanostructures along programmed creases, i.e., folding lines. Based on the characteristic of high flexibility compared to the dense and flat structure, in the present invention, a wireframe nanostructure composed of DNA segments was designed to mimic a macroscopic paper called ‘DNA origami paper (or DNA paper)’ (FIG. 1A). In order to implement various nanoscale creases through DNA origami paper (FIG. 1B), the present inventors have developed a platform-based and modular creases programming method (FIG. 1C).

In this method, two types of crease handles of 3′ terminal and 5′ terminal were designed protruding from the DNA segment. In the drawings of this specification, semi-circular arrows on DNA papers were indicated in the direction form the 3′ crease handles to the 5′ crease handles. The protruding portion of the crease handle consists of 8-nt long single-stranded DNA (ssDNA) for binding, and 3-nt poly-T for spacers. Adding the glue strands activates the folding of the DNA paper through a base pair which is complementary to two crease handles. The glue strands were designed to have a 5-nt long toehold at the terminal thereof. Therefore, when a releaser strand having a sequence completely complementary to the glue strand is introduced, the folded structure may be returned to its initial unfolded shape through toehold-mediated DNA displacement.

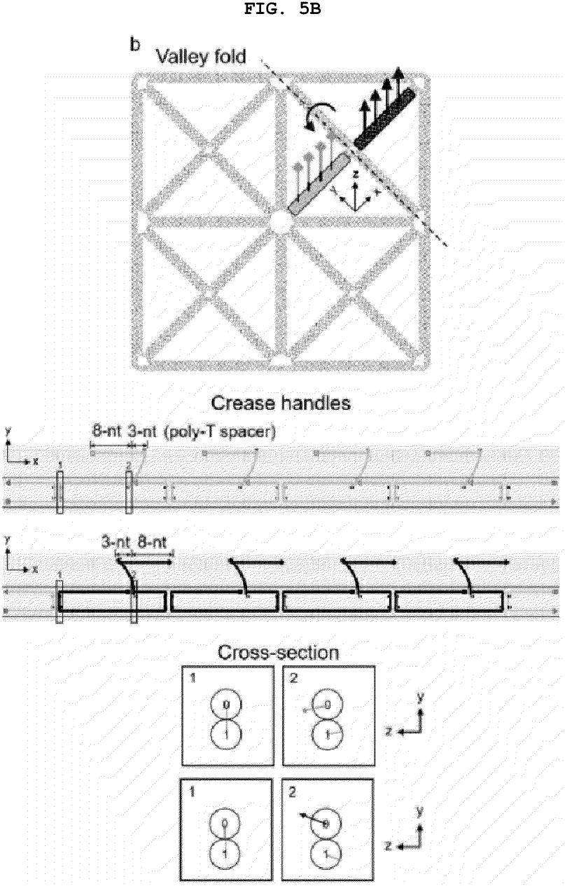

The present inventors first demonstrated a crease programming method through half folding and unfolding of the square-shaped DNA origami paper (FIG. 1A to FIG. 1F). A draft of the DNA paper structure was designed using PERDIX, and a pair of DNA segments arranged on both sides of a target crease were modified to have six 3′ and 5′crease handles, respectively (FIG. 5A and FIG. 5B).

After annealing the DNA paper programmed with a half folding crease pattern based on this design, the remaining staples were removed through the ultrafiltration, and diluted by titration (left in FIG. 1D). Glue strands were added in the order of 10 times the number or concentration of the crease handle pairs (middle in FIG. 1D) , and in this case, 6 pairs and releaser strands were added in the order of 10 times the number or concentration of the added glue strands (right in FIG. 1D). Through atomic force microscopy (AFM) , it was possible to observe that the half folding and unfolding of the DNA paper was implemented as intended. The half folded DNA paper measured at 2% both before adding the glue strands and after adding the releaser strands, but almost 60% was observed after adding the glue strands (see FIG. 6A and FIG. 6B for the full AFM image). It was also possible to confirm the measured height of five peak points (arrows in the FIG. 1D) of the square DNA paper. At first, the height was between 0.6 nm and 0.9 nm, but after the addition of glue strands (arrows in the FIG. 1E), the height was exhibited to be 1.2 nm or more, about 2 times higher than before the addition. They were also measured to their original heights after adding the releaser strands (arrows in the FIG. 1F). These results show that the DNA paper can be folded and unfolded along the programmed creases.

### 2. Implementation of Various DNA Origami

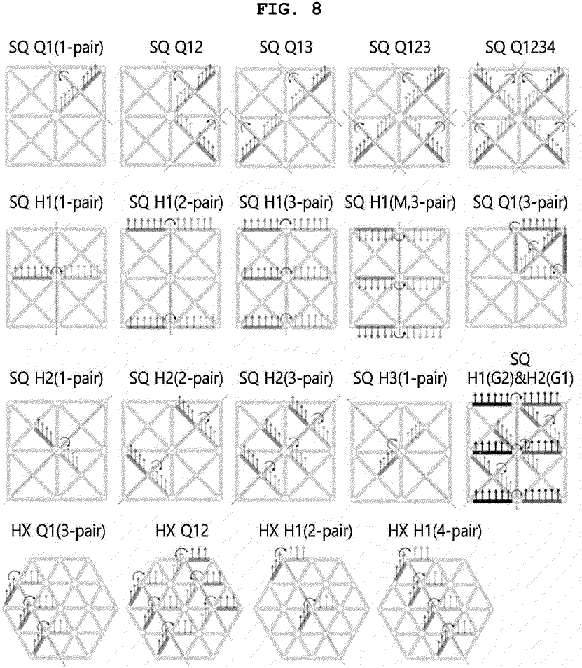

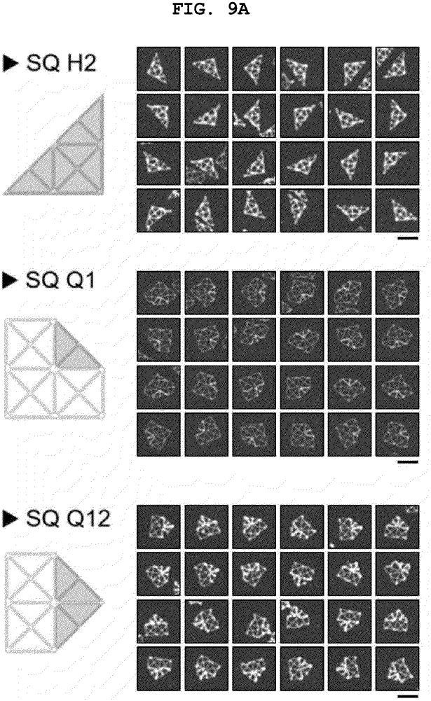

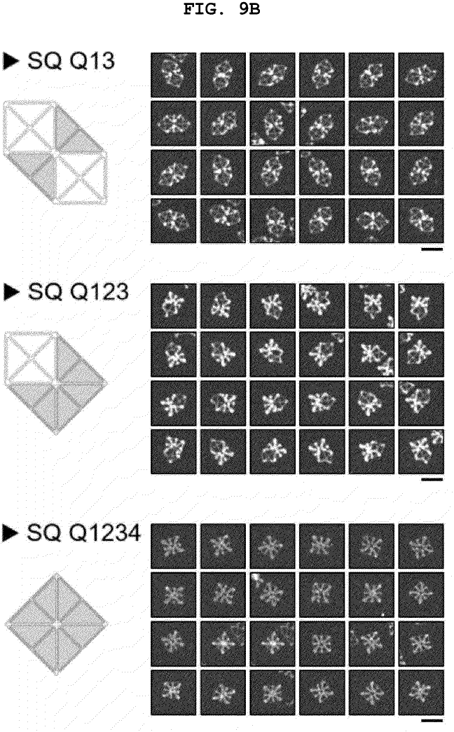

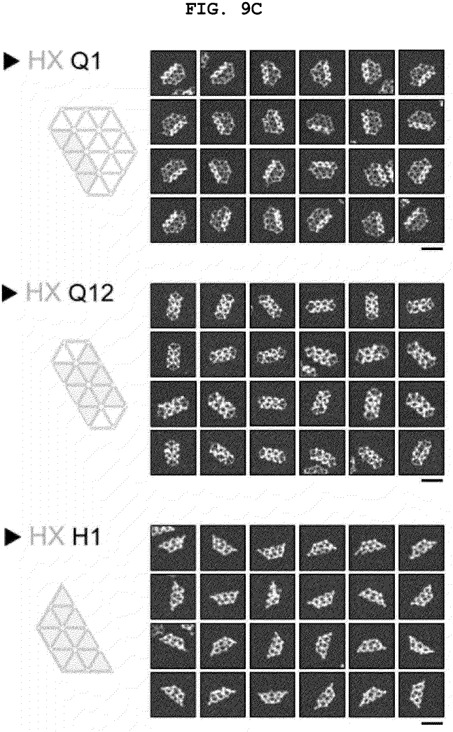

Based on the experimental results of the half folded origami, nine creases were further designed on two types of DNA paper for the existing square (SQ) and newly added hexagonal (HX) shapes (FIG. 7A to FIG. 7E, FIG. 2A, FIG. 2B and FIG. 8). According to the folding type, the crease was named such as prefix H for half (½) fold, and prefix Q for quarter (¼) fold, etc., and the number of the suffixes was used to distinguish the same fold. Each crease was modularly modified and programmed so that the DNA line segments located on both sides of the target crease had crease handles(FIG. 7A to FIG. 7E) . AFM measurements were performed on all creases, and the measured results show that it is possible to program nanoscale origami through the DNA paper (FIG. 9A to FIG. 9C). Further, in order to compare the equilibrium shape of the folded DNA paper with the experimental results, finite element analysis was performed using SNUPI to which the division and rearrangement method were applied (FIG. 7A to FIG. 7E, and FIG. 10A to FIG. 10C). In agarose gel electrophoresis, the various degrees of migration were shown depending on the folded shape, and in general, the more folded, the higher the band mobility (FIG. 7A to FIG. 7E and FIG. 11). Such a difference in overall mobility indicates that the DNA paper was intentionally folded along its own target creases in a solution rather than accidentally folded due to a two-dimensional projection problem on the mica during the AFM measurements.

### 3. Optimization of Origami

Although it was experimentally confirmed that the DNA paper was folded with a certain yield according to the programmed creases, the estimated single origami yield based on the AFM images was still less than 70%, which was not enough to implement more complex origami and its properties on the nanoscale (see experimental method for yield estimation process). Therefore, in order to achieve an optimal origami yield, two origami motifs, pairs of creases and gaps, were devised and applied to the present invention in combination.

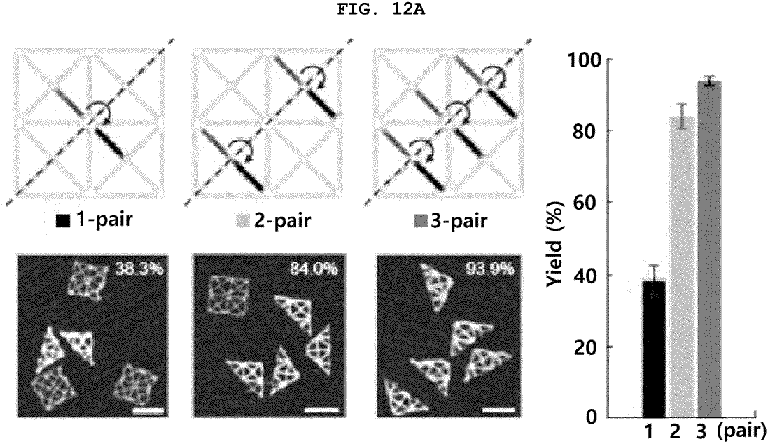

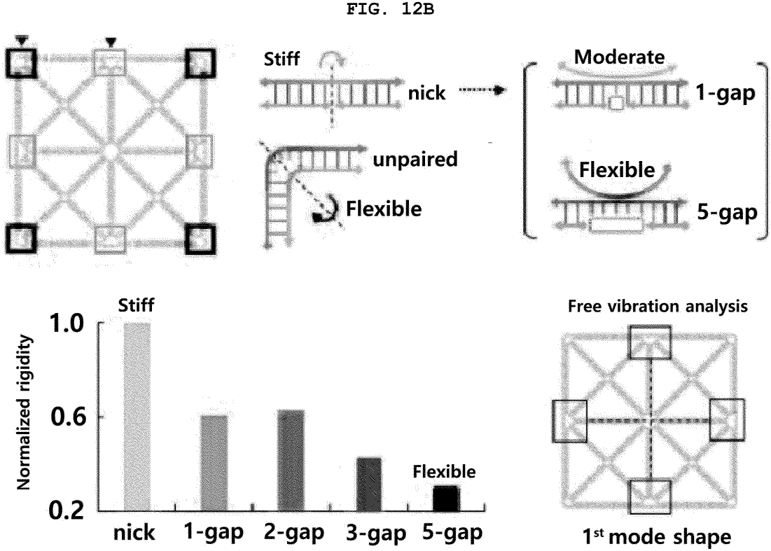

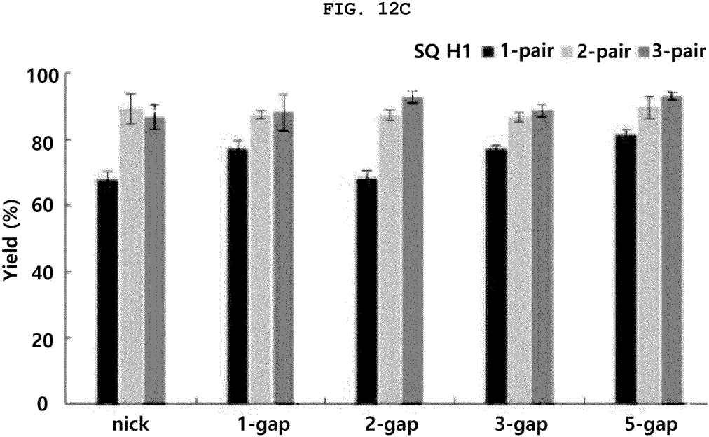

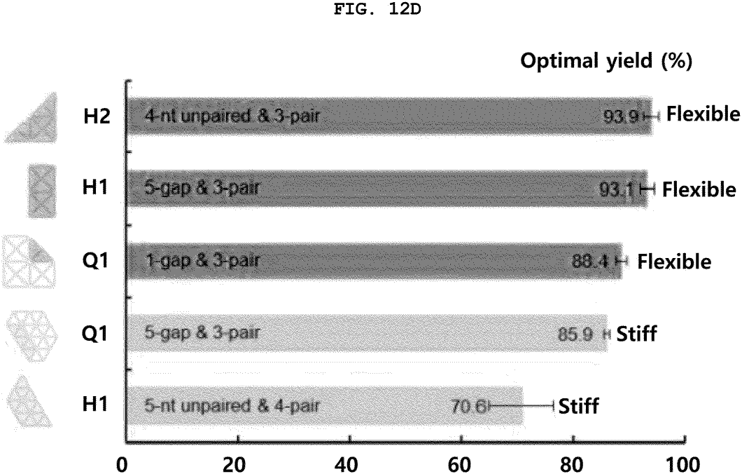

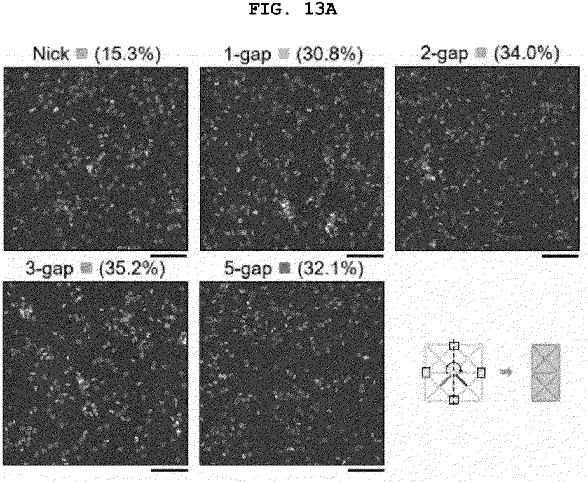

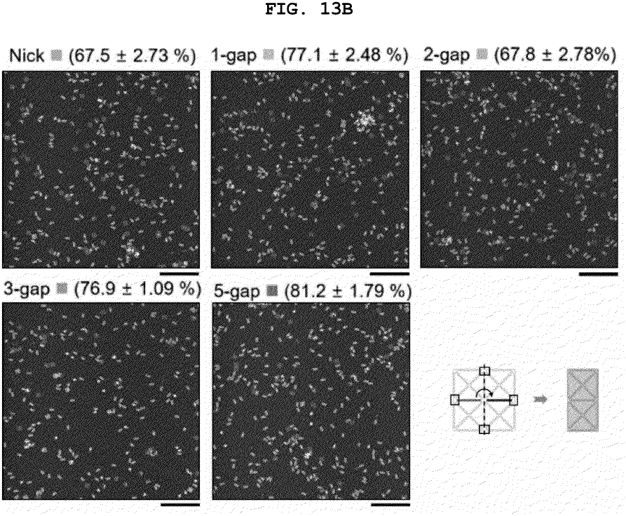

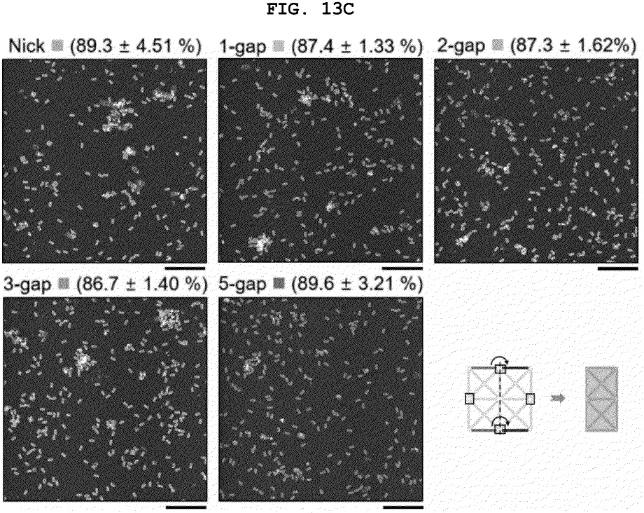

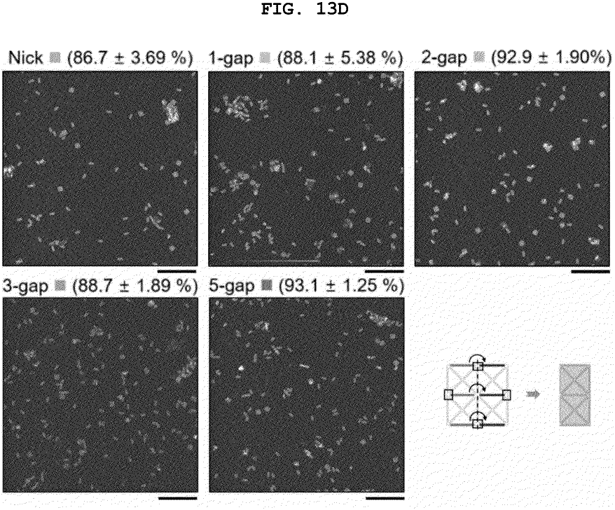

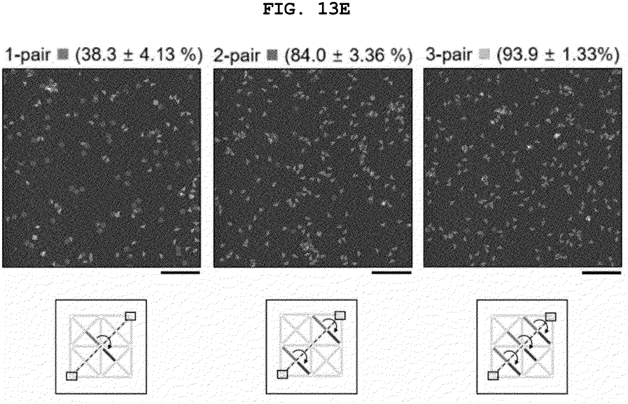

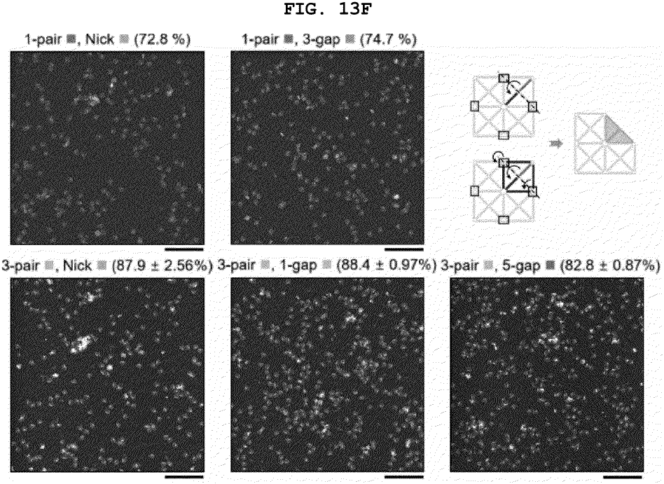

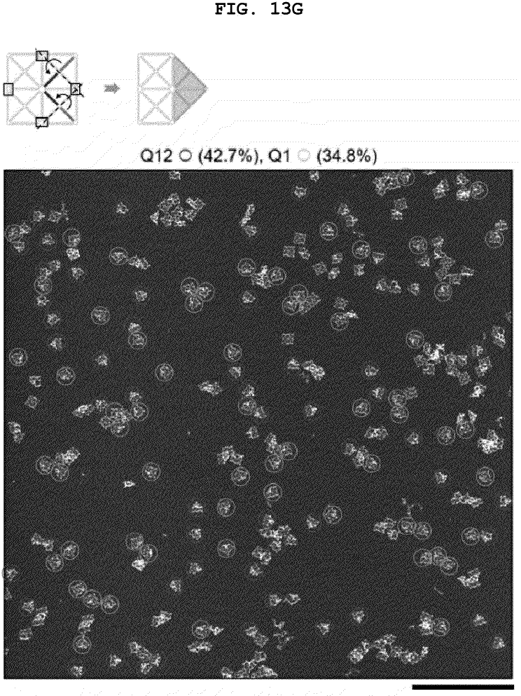

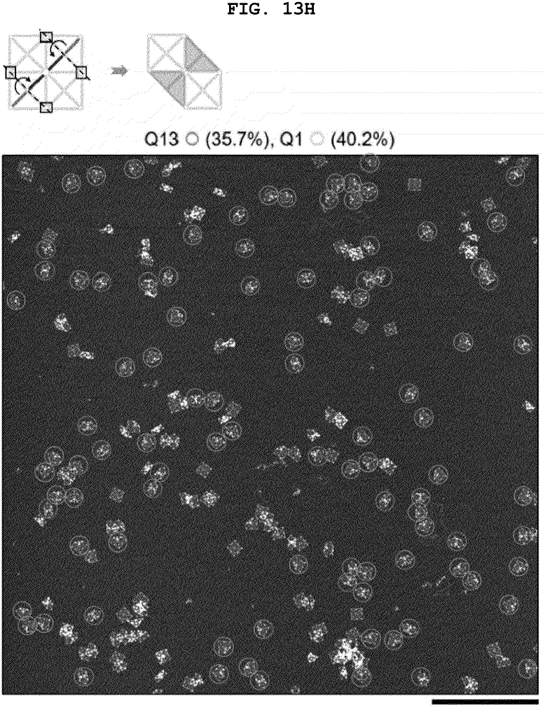

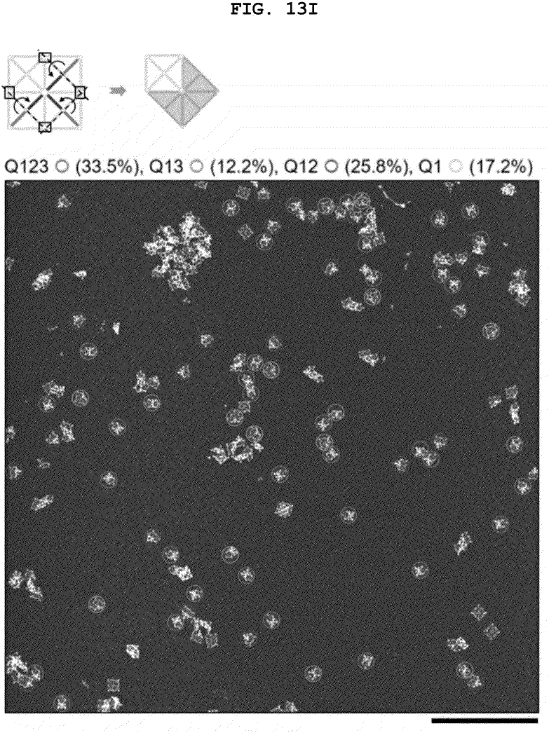

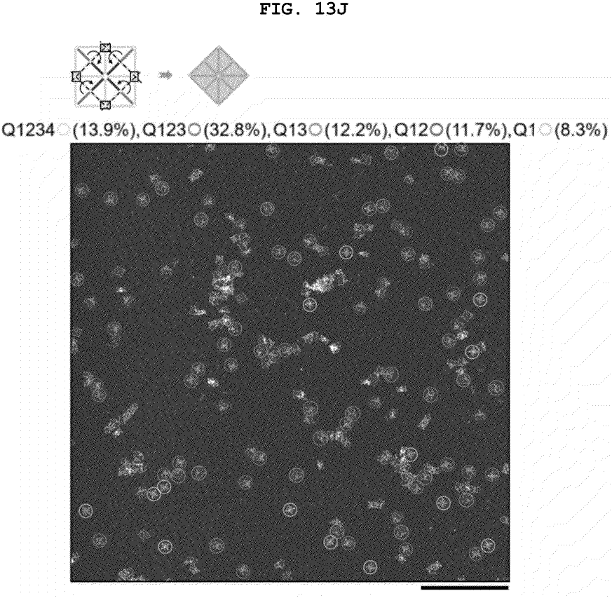

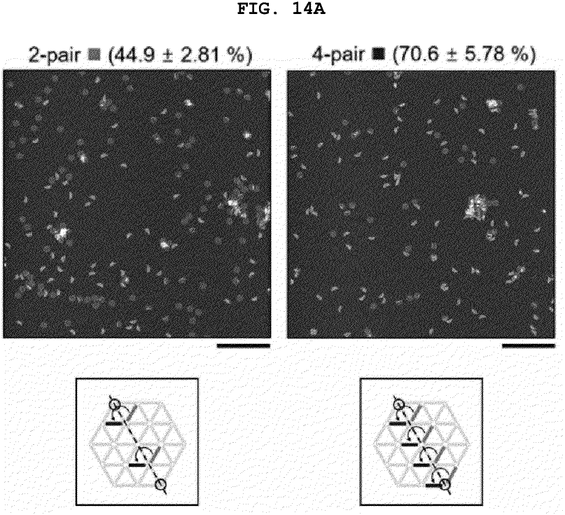

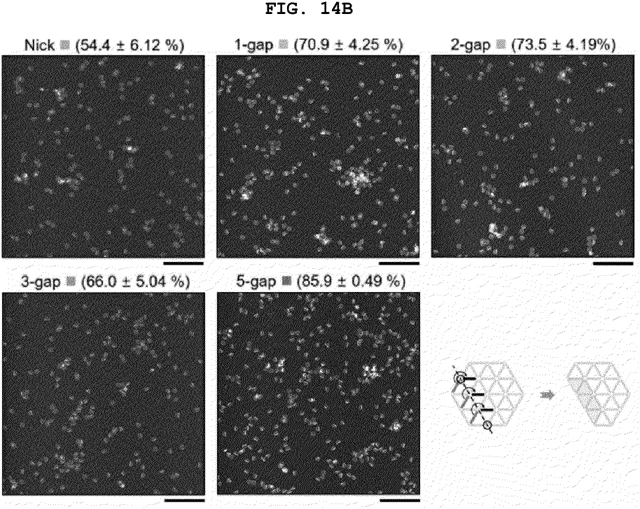

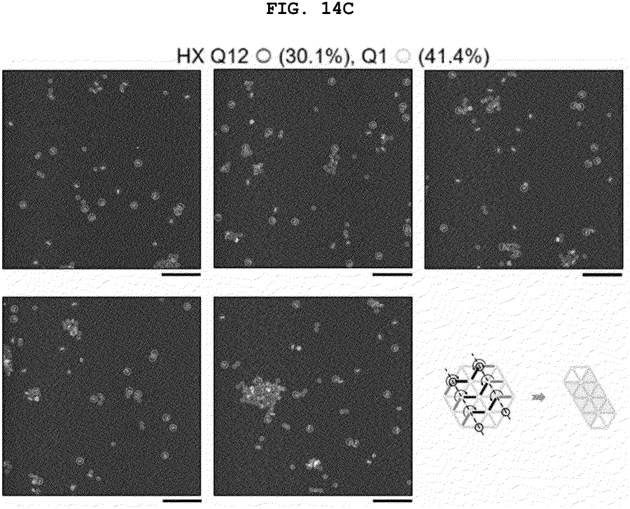

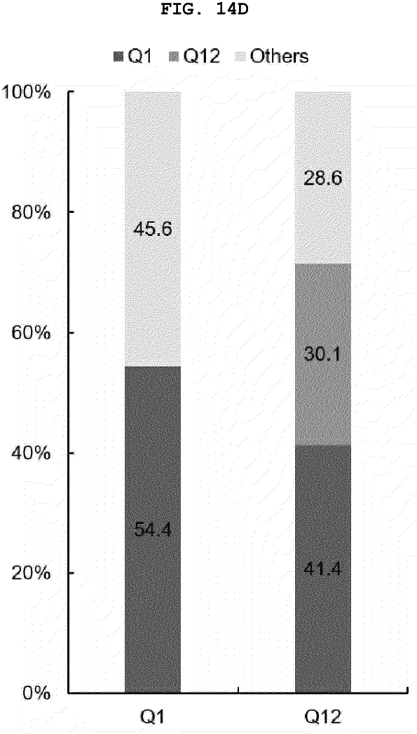

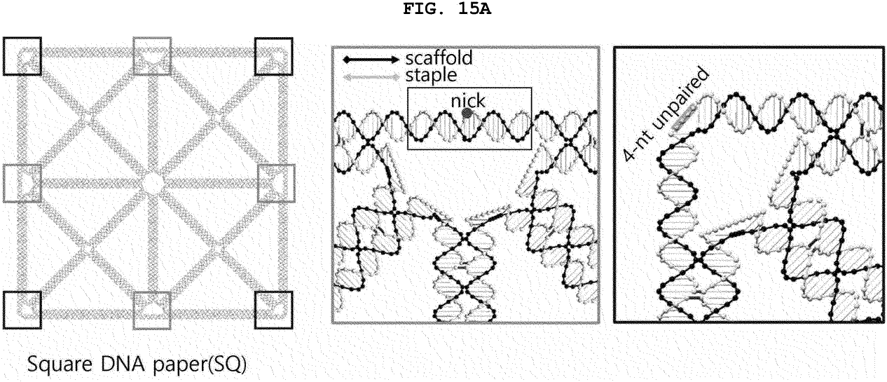

First, in order to increase the folding probability of the DNA paper, the number of crease pairs was increased along the target crease (FIG. 12A to FIG. 12D). Each origami yield was experimentally calculated and compared through AFM images. For SQ H2, the origami yield was increased from 38.3% of one pair to 84.0% of two pairs, and finally increased to 93.9% of three pairs (FIG. 13E). Other origami shapes were also tested to confirm whether they are applied well as in the normal case. Similarly, it was observed that the origami yield of SQ Q1 was improved from 72.8% of 1 pair to 87.9% of 3 pairs, and SQ H1 was improved from 67.5% of 1 pair to 86.7% of 3 pairs (FIGS. 13B to 13F). In the case of HX, 4 pairs of HX H1 showed a yield of 70.6%, which was about 25% higher than that of 2 pairs (FIG. 14A). This result suggests that the number of crease pairs is a major factor contributing to an increase in the origami yield.

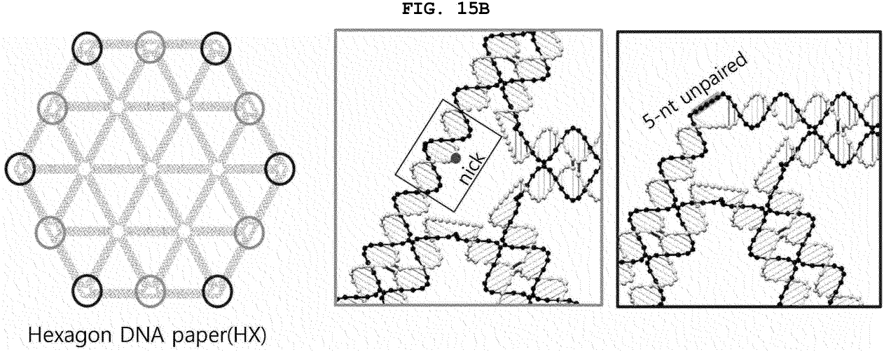

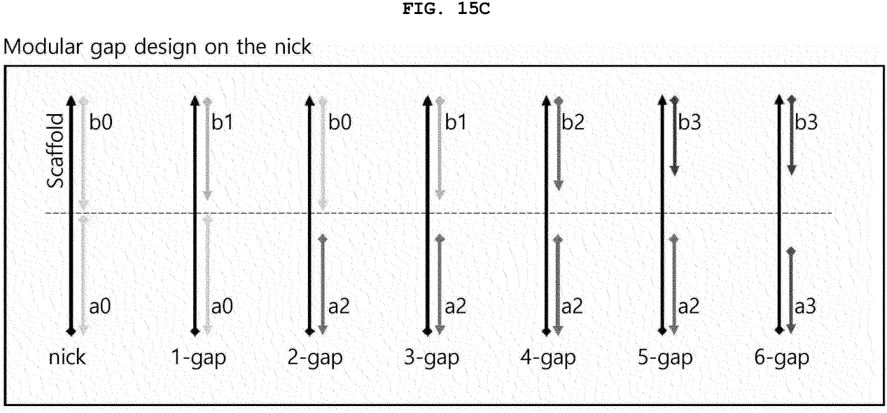

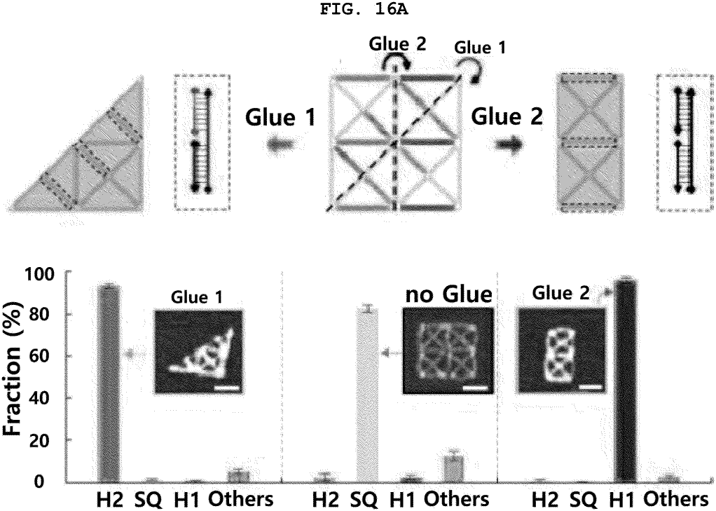

Next, it was confirmed that 4 out of 8 vertex regions of the square DNA paper consisted of nick regions (light boxes in FIG. 12B) where all bases are fully paired and one phosphate-based DNA skeleton is locally broken. On the other hand, the remaining four vertex regions had unpaired regions (dark boxes in FIG. 12B), which had no self-complementary bases without skeleton breakage. By applying a local gap in the nick region, it was expected to induce a decrease in the bending rigidity, which has finally resulted in an improvement in the origami yield. Corresponding nicks were modified with a gap having various lengths ranging from 1-gap to 5-gap by modularly changing the apical staples of caDNAno (FIG. 15A to FIG. 15C). First, in order to theoretically confirm effects, free vibration analysis of plate theory was performed using SNUPI based on the finite element framework, and finally, the flexural rigidity of DNA paper having various gaps was estimated computationally. As expected, as the length of the gap was increased, the normalized flexural rigidity was decreased as a whole, and the value of 5-gap was finally measured to be about 30% of the nick. Therefore, it was shown that the overall rigidity of the DNA paper could be relaxed by applying a gap to the nick. This suggested that the folding yield could be increased through the gap.

In order to experimentally investigate the effect obtained by the number of pairs of creases and the number of gaps, which are two origami motifs, a total of 15 cases of SQ H1 were designed by varying the number of pairs and gaps (FIG. 12A to FIG. 12D, and FIG. 13B to FIG. 13D) . Similarly to the previous results for crease pairs, the overall origami yield for most gaps was higher as more crease pairs were designed. In the case of 1-pair, the yield was not increased monotonically as more gaps were applied, but the folding yield was finally improved from 67.5% to 81.2% by applying 5-gap to the existing nick. When applying two or more pairs of creases, the overall origami yield was increased to 86% or more regardless of the number of gaps, and 93.1% of the highest yield was reached in 5-gap and 3-pair. For all three pairs, the highest yields were shown to be 81.2% (1-pair), 89.6% (2-pair), and 93.1% (3-pair) when 5-gap. Through this, it was confirmed that the number of the designed crease pairs is the most dominant factor in the folding yield, and it may be further optimized through the gap.

Finally, the optimal yield for different cases of single-folded origami was investigated based on the results obtained by controlling the crease pair and the gap motif together. SQ was measured to be 88.4% of Q1 (3-pair and 1-gap), 93.1% of H1 (3-pair and 5-gap), and 93.9% of H2 (3-pair and 4-nt unpaired), while HX was measured to be 70.6% of H1 (4-pair and 5-nt unpaired), and 85.9% of Q1 (3-pair and 5-gap) (FIG. 12A to FIG. 12D, FIG. 13D to FIG. 13F, FIG. 14A and FIG. 14B). The overall optimal yield of HX was calculated to be smaller than that of SQ, and the relatively denser and more stable internal mesh structure of HX was assumed to be a factor. The reason is that HX includes 28 inner triangles more than 16 of SQ, and has a total area 10% larger than SQ.

### 4. Origami Properties

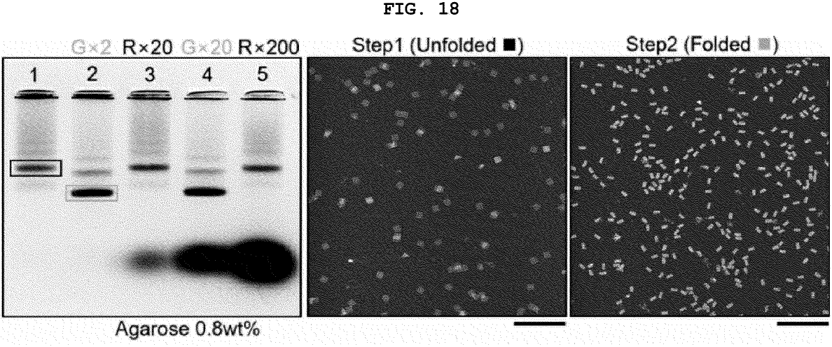

After achieving the optimal origami yield of the DNA paper, various origami properties were searched for on the nanoscale, and a first target was an orthogonal-type origami. SQs having two orthogonal crease patterns H1 and H2 were designed by adding new glue strands (glue 2) of a sequence orthogonal to the newly designed two types of crease handles (horizontal line segments on the DNA paper in the drawing) (FIG. 16A to FIG. 16D). The most dominant state before adding the glue strands is the unfolded state (SQ) at a rate of 82.6% in the AFM images (FIG. 17). The dominant configuration was converted to 93% of the H2 state by adding the glue 1 strands, while it was changed to 96% of the H1 state by glue 2 strands in the unfolded SQ. When the opposite glue strands were introduced, that is, both ratios of H1 in glue 1 and H2 in glue 2 were measured to be less than 1%. Further, in order to investigate the reason why the unfolded ratio in the absence of glue strands is 80%, which is 10% lower than the folded ratio of 90%, the remaining ratio (“Others” bars) with no glue strand was analyzed. In the absence of glue strands, the ratio of the remainder was, on average, 10% higher than in the presence of the glue strands. Among them, the ratio of Q1, one of the shapes included in the remainder, accounted for 9.6% with no glue strand, but was decreased to less than 1% after adding glue 1 or 2 strands. Through this, the present inventors have found that DNA papers which appeared in a partially folded shape during deposition on mica for AFM measurement resulted in a 10% lower ratio to a fully unfolded shape, and after adding the glue strands, this possibility is decreased due to the reduced projection area, such that a 90% folding ratio, which is 10% higher, was achieved. Based on these orthogonal characteristics, it is expected that a more complex orthogonal origami may be implemented in the future by designing an orthogonal design having more crease handles and glue strands.

Next, the present inventors have studied repeatable DNA origami using H1 crease patterns, and expected that the DNA paper would be folded and unfolded sequentially by alternately introducing the glue strands and releaser strands. To confirm the overall shape change, agarose gel electrophoresis was performed to compare the degree of migration. The initial band position of the unfolded state (U) was lowered to a lower position of the folded state (F) as the glue strands were introduced, and the two positions were repeated according to the folded and releaser strands introduced therein. To further verify that the DNA paper of each band had the intended shape, folded or unfolded shape, the bands were carefully extracted using a razor blade, then the samples were filtered and AFM measurements were performed (FIG. 18). In the U band, 4% of the H1 state and 81% of the unfolded state were observed, and in the F band, the H1 state was 97.6%, and the unfolded state was not observed.

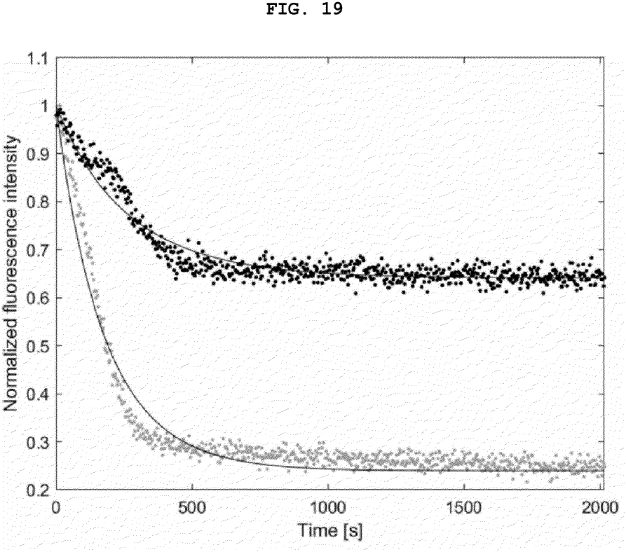

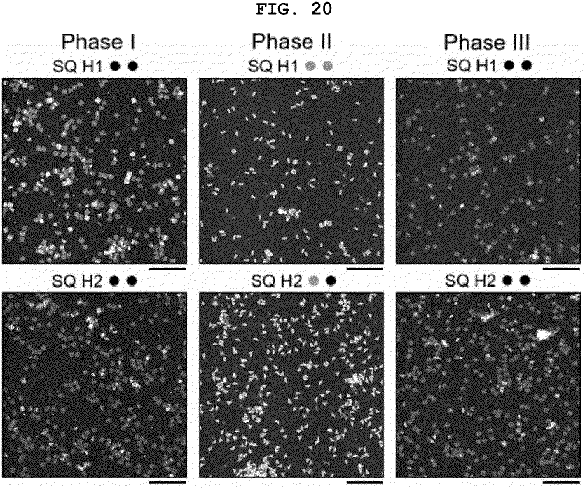

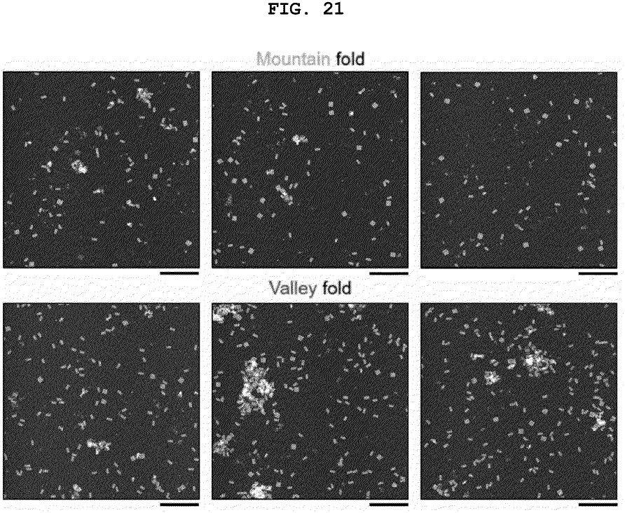

In addition, by designing two quenchers (Q, and EBQ) and fluorescent reporters (R, and Cy3) at four inner vertices of SQ, a change in the origami-dependent fluorescence intensity through proximity-induced quenching effect was predicted (upper portion in c of FIG. 4B). Two types of DNA paper having H1 and H2 crease patterns, respectively, were designed to have two reporters and quenchers at the four inner vertices (FIG. 4A and FIG. 4B), and kinetics for the following three steps were measured for a total of 140 minutes while adding the glue strands or releaser strands, which includes: Phase I of an initial state; Phase II of folding state, and Phase III of unfolding state (FIG. 16A to FIG. 16D). All data were normalized to compensate for the dilution effect, and were initially maintained at an intensity of about 1.0 with slight fluctuations (Phase I). After adding the glue strands, the normalized intensities of H1 and H2 were decreased to near 0.25 and 0.65, respectively, and finally converged (Phase II) . Both origami took about 10 minutes to reach convergence, and their velocity was estimated to be up to 4.3 × 10⁵ (M · s)⁻¹ in the kinetic model of the present invention (solid lines in FIG. 16A to FIG. 16D and FIG. 19). The intensity started to be increased again after adding the releaser strands, and then was recovered and converged to almost 0.95 (Phase III). The unfolding reaction took about 40 minutes, thus being much slower to reach convergence than the folding reaction. This seems to be a reasonable result because the toehold-mediated DNA displacement reaction is generally slower than the binding of single-stranded DNAs, in this case, binding between the glue strands and the crease handles. In addition, AFM was measured to further verify the origami shape of the DNA paper at each phase, and it was possible to observe that DNA paper was folded and unfolded as intended (FIG. 16A to FIG. 16D and FIG. 20).

Finally, an experiment was performed to confirm whether origami in two opposite directions of the mountain and valley shapes could be implemented along the same crease handle. The existing fold was set as the fold of the valley portion, and a crease handle in the direction opposite to the existing protruding direction was newly designed, then this was set as the fold of the mountain portion (FIG. 16D, see FIG. 5A and FIG. 5B for detailed crease patterns). Since the SQ H1 crease pattern was used, and it was designed so that two reporters and quenchers were attached to ends of the vertex strands protruding in the existing valley fold direction, it was expected that a relatively higher fluorescence intensity would appear in the mountain-shaped fold having low proximity between the quencher and reporter than in the valley-shaped fold. When the fluorescence intensity of the mountain creases was converged after the addition of the glue strands, kinetics were measured for 15 minutes and compared with those of the valley creases. The overall normalized fluorescence intensity in the mountain-shaped fold was consistently slightly higher than the fluorescence intensity in the valley-shaped fold as expected. In addition, by performing an AFM experiment, it was confirmed whether the shape was the intended origami, and it was confirmed that the origami was folded in half, which is the intended shape, with high yield (FIG. 21). The origami yield estimated by AFM was 95% or more, which was slightly higher than the previous ones, and it seems to be because the internal temperature of the spectrofluorometer during the measurement was higher than room temperature.

What is claimed is:

1. A method for preparing a nucleic acid structure, the method comprising:
binding at least a pair of first single-stranded nucleic acids protruding in the same direction to portions symmetric with respect to at least one crease line segment, respectively, in a nucleic acid wireframe having a plurality of line segments; and
treating the nucleic acid wireframe with a second single-stranded nucleic acid which includes a first portion including a complementary sequence to the pair of first single-stranded nucleic acids and a second portion including a non-complementary sequence to the pair of first single-stranded nucleic acids at a terminal of the first portion, thus to bind the pair of first single-stranded nucleic acids to the second single-stranded nucleic acid.

2. The method of claim 1, wherein the pair of first single-stranded nucleic acids consist of two single-stranded nucleic acids having different sequences.

3. The method of claim 1, wherein, in the binding of the at least a pair of first single-stranded nucleic acids, the pair of first single-stranded nucleic acids are bound orthogonal to the portions symmetric with respect to the at least one crease line segment in the same direction.

4. The method of claim 1, wherein the at least one crease line segment comprises a plurality of crease line segments; and
the pair of first single-stranded nucleic acids have different sequences for each of the plurality of crease line segments.

5. The method of claim 1, wherein, in the binding of the at least a pair of first single-stranded nucleic acids, a plurality of the pair of first single-stranded nucleic acids are bound to the symmetrical portions, respectively; and
in the treating of the nucleic acid wireframe, the treating is performed so that a plurality of second single-stranded nucleic acids are bound to the first single-stranded nucleic acids as many as the number thereof.

6. The method of claim 1, wherein, in in the binding of the at least a pair of first single-stranded nucleic acids, a pair of first single-stranded nucleic acids on both sides of the symmetrical portion are bound to the first portion of the second single-stranded nucleic acid, respectively.

7. The method of claim 1, further comprising binding a fluorescent material and a quencher respectively to both sides of the wireframe so as to be symmetric with respect to the at least one crease line segment, before the treating of the nucleic acid wireframe.

8. The method of claim 1, further comprising adding a third single-stranded nucleic acid which includes a complementary sequence to the first portion and the second portion, thus to bind the second single-stranded nucleic acid to the third single-stranded nucleic acid so as to be separated from the first single-stranded nucleic acid.

9. A method for detecting a target nucleic acid, the method comprising:
binding at least a pair of first single-stranded nucleic acids protruding in the same direction to portions symmetric with respect to at least one crease line segment, respectively, in a nucleic acid wireframe having a plurality of line segments; and
treating the nucleic acid wireframe with a sample suspected of containing a target nucleic acid which includes a first portion including a complementary sequence to the pair of first single-stranded nucleic acids and a second portion including a non-complementary sequence to the pair of first single-stranded nucleic acids at a terminal of the first portion, thus to confirm whether the wireframe is folded at the line segment.

10. The method of claim 9, wherein the pair of first single-stranded nucleic acids consist of two single-stranded nucleic acids having different sequences.

11. The method of claim 9, wherein the at least one crease line segment comprises a plurality of crease line segments; and
the pair of first single-stranded nucleic acids have different sequences for each of the plurality of crease line segments.

12. The method of claim 11, wherein the target nucleic acid comprises a plurality of nucleic acids having different sequences.

13. The method of claim 9, further comprising binding a fluorescent material and a quencher respectively to both sides of the wireframe so as to be symmetric with respect to the at least one crease line segment, before the treating of the nucleic acid wireframe.

14. The method of claim 9, wherein, when the line segment is folded, it is determined that the target nucleic acid corresponding to the pair of first single-stranded nucleic acids which are bound corresponding to the at least one crease line segment is present in the sample.

15. The method of claim 14, wherein, when a fluorescence intensity of the fluorescent material is decreased, it is determined that the line segment is folded.

16. The method of claim 13, wherein the at least one crease line segment comprises a plurality of crease line segments; and
an amount of the target nucleic acid in the sample is quantified based on a degree of a decrease in the fluorescence intensity of the fluorescent material.

17. A nucleic acid structure comprising:
a nucleic acid wireframe having a plurality of line segments; and
at least a pair of first single-stranded nucleic acids which protrude in the same direction and are bound to portions symmetric with respect to at least one crease line segment in the nucleic acid wireframe,
wherein, among a first portion and a second portion adjacent to each other in a target nucleic acid sequence, the pair of first single-stranded nucleic acids have a complementary sequence to at least a part of the first portion and a non-complementary sequence to the second portion.

18. The nucleic acid structure according to claim 17, wherein the pair of first single-stranded nucleic acids consist of two single-stranded nucleic acids having different sequences.

19. The nucleic acid structure according to claim 17, wherein the nucleic acid wireframe includes a DNA origami wireframe.

20. The nucleic acid structure according to claim 17, wherein a fluorescent material and a quencher are further bound respectively to both sides of the wireframe so as to be symmetric with respect to the crease line segment.
